(19) United States
(12) Patent Application Publication
(10) Pub. No.: US 2011/0118194 A1
(43) Pub. Date: May 19, 2011

(54) Therapeutic Use of LPI, a Staphylococcal Lectin Pathway Inhibitor in Inflammatory Diseases

(75) Inventors: Willem Jan Bastiaan VAN WAMEL, Utrecht (NL); Suzan Huberdian Maria Rooijakkers, Utrecht (NL); Cornelis Petrus Maria Van Kessel, Bunnik (NL); Johannes Antonius Gerardus Van Strijp, Odijk (NL)

(73) Assignee: UMC Utrecht Holding B.V., CM Utrecht (NL)

(21) Appl. No.: 12/939,872

(22) Filed: Nov. 4, 2010

Related U.S. Application Data
(62) Division of application No. 10/561,583, filed on Mar. 15, 2007, now abandoned.

(30) Foreign Application Priority Data
Jul. 8, 2003 (EP) 03077138.0

(51) Int. Cl. A61K 38/16; A61K 38/02

(52) U.S. Cl. 514/21.2; 514/1.1

(57) ABSTRACT

The invention relates to nucleic acid molecules encoding (poly)peptides having LPI (Lectin Pathway Inhibitor) activity, to recombinant vectors harboring such molecules, and the host cells carrying the vectors. The invention further relates to methods for preparing recombinant (poly)peptides having LPI activity and to the use of such recombinant (poly)peptides having LPI activity for diagnosis, prophylaxis and treatment, such as the treatment of inflammation reactions. In addition the invention provides therapeutic and diagnostic compositions comprising as the active ingredient the (poly)peptide having LPI activity.

The present invention relates to a (poly)peptide having complement inhibitory activity. The invention further relates to a nucleic acid molecule encoding this (poly)peptide and the use of the information contained in the nucleic acid for the preparation of the corresponding (poly)peptide and to vectors and hosts for use therein. The invention in addition relates to non-(poly)peptide molecules having a similar structure and function as the (poly)peptides. The (poly)peptide having Lectin Pathway Inhibitor (LPI) activity that is encoded by the nucleic acid molecule of the invention can be used in the treatment of inflammatory reactions. The (poly)peptides and non-(poly)peptides can in addition be used for inhibiting activation of complement.

Complement is the complex network of over 20 serum proteins that are part of our innate immune system. Complement acts by itself (through lysis of microbes) or in conjunction with other components of the innate immune system (e.g. phagocytosis). Our innate immune system is mainly involved in protecting the body against foreign invaders (e.g. bacteria, viruses, fungi, and also cancer cells). The most important cells of the innate immune system are dendritic cells, monocytes/macrophages and neutrophils. Next to that, our innate immune system contains a large variety of soluble factors such as acute phase proteins, antimicrobial peptides, peptidases, parts of the clotting cascade and the complement system. Killing and removal of invaders is mostly done by monocytes and neutrophils, by direct recognition of the invaders or with the help of specific antibodies and/or the complement system (opsonization).

Cells of the innate system react in a relatively aggressive way. Since they are part of the body's first line of defense, their most important task is to kill and remove the invading agent as quickly as possible. This is accomplished through very aggressive substances (e.g. free radicals and enzymes) that are not only lethal to the invader, but also cause damage to host cells in the vicinity. Substances from these damaged cells and the locally activated cells from the innate system itself will further attract increasing numbers of neutrophils and monocytes, causing further local inflammation.

In most cases, such an aggressive and damaging inflammatory reaction, caused by over-activated neutrophils, is unnecessary. In some cases this inflammatory response is responsible for serious, sometimes lethal disorders and includes conditions like Adult Respiratory Distress Syndrome (ARDS), severe tissue damage following thrombotic events such as heart attacks and stroke, inflammatory bowel diseases and rheumatoid arthritis.

The inflammation will subside once all the invaders have been killed and removed, together with the various cells killed in the process. Healing of the wound, caused by the inflammatory response, can then begin. Although there is some overlap in function, the main task of neutrophils is to attack the invaders and the main task of monocytes is to remove the debris resulting from this attack. In addition, neutrophils have another peaceful task in assisting the wound healing process.

When bacteria have invaded the body, substances of microbial origin activate the complement system directly or via pre-existing antibodies. The first molecule involved in antibody-mediated complement activation is C1q followed by the activation of C1r and C1s. A parallel pathway does not need specific antibodies, because it directly recognizes microbial surface structures. H-ficolin, L-ficolin or Mannose Binding Lectin (MBL) recognize microbes and through activation of a specific MBL Associated Serine Proteases (MASP-2), the rest of the complement system is activated.

In both events activation proceeds through C4 and C2 and the central molecule of the complement system C3 is activated. This leads to more C3 deposition via the alternative pathway (factors B, D, H, I, P). C3 once converted into C3b, C3bi or even C3d is the most important opsonin, it mediates uptake of microbes by phagocytes, and importantly also activates these phagocytes in the process. Next to this phagocyte directed action, the complement system can proceed from C3 via C5, C6, C7, C8, and C9 to lysis of the tumor cell, virus infected cell, gram negative bacterium, or during unwanted inflammatory events, one of the healthy cells of our body.

Normally our cells are protected from unwanted complement attack by a variety of mechanisms (C1INH, C4 bp, CR1, MCP, DAF, H, I, P, CD59) but in cases of disturbance or extremely high local activation, direct complement mediated damage can still occur. Furthermore, in the process of complement activation, the formation of opsonins and membrane attack is parallelled by the formation of very strong inflammatory small molecules (C5a, C3a). These substances directly activate phagocytes and other cells (via C5a and C3a receptors) in a very efficient way, resulting in damage to microbes or healthy cells. The interaction with different cell types also gives rise to the production of other chemokines (like interleukin-8, IL-8): substances that can activate and attract cells from the blood vessels (the migration process). Neutrophils interact with these substances, because they have receptors for these substances on the outside of their cell membrane. An overview of the components of the complement system is given in Table 1.

TABLE 1
PROTEINS INVOLVED IN THE COMPLEMENT CASCADE

| | |
|---|---|
| Binding to Ag:Ab complexes: | C1q |
| Direct recognition of | MBL, Ficolin-H, L-Ficolin, |
| microbial surface structures: | M-Ficolin |
| Activating Enzymes: | C1r, C1s, C2b, B, D, MASP1, 2, 3 |
| Membrane-binding opsonins: | C4b, C3b |
| Mediators of inflammation: | C5a, C3a, C4a |
| Membrane attack: | C5b, C6, C7, C8, C9 |
| Complement Receptors: | CR1, CR2, CR3, CR4, C1qR, M-Ficolin |
| Complement-regulatory | C1INH, C4bp, CR1, MCP, DAF, H, I, |
| proteins: | P, CD59 |

*Adapted from Janeway & Travers Immunobiology, 1996; Current Biology Ltd/Garland Publishing Inc.

Activated neutrophils can easily migrate from blood vessels. This is because the chemokines and microbial products will have increased the permeability of the vessels and stimulated the endothelial cells of the vessel walls to express certain adhesion molecules. Neutrophils express selectins and integrins (e.g. CD11b/CD18) that bind to these adhesion molecules. This process is called priming. Once the neutrophil has adhered to the endothelial cells, it is able to migrate through the cells, under the guidance of chemokines, towards the site of infection, where the concentration of these substances is at it's highest.

These substances also activate neutrophils to produce a range of other molecules, some of which attract more neutrophils (and subsequently monocytes), but, mostly, they are responsible for destroying the invading bacteria. Some of these substances (e.g. free radicals, enzymes that break down proteins (proteases) and cell membranes (lipases)) are so reactive and non-specific that cells from the surrounding tissue (and the neutrophils themselves) are destroyed, causing tissue damage. This damage is exacerbated by the presence of blood-derived fluid, which has transgressed the leaky vessel wall and is responsible for the swelling that always accompanies inflammation (called edema). The pressure build up caused by this excess fluid results in further cell damage and death.

The onset of an inflammatory reaction does not have to be of microbial origin per se. Tissue damage in general, by oxygen deprivation, pH changes, salt disbalance or physical damage can start inflammatory reactions. In many cases the key event is the activation of the complement system. In autoimmune diseases, the presence of auto-antibodies gives rise to the activation of the classical pathway of complement. In almost all other events the activation of complement is via the lectin pathway (cf. Jordan et al., Circulation. 2001, 104(12):1413-8; Collard et al., Am J. Pathol. 2001, 159:1045-54; Roos et al., J. Immunol. 2001, 167:2861-8; Collard et al., Am J Pathol. 2000, 156:1549-56; Collard et al., Mol Immunol. 1999, 36:941-8). Therefore the lectin pathway of complement activation is of crucial importance as the first step in many inflammatory conditions and diseases.

Later in the inflammatory process, monocytes migrate to the scene and become activated. Besides their role in removing bacteria and cell debris, they also produce substances such as tumor necrosis factor (TNF) and IL-8, which in turn attract more activated neutrophils, causing further local damage. TNF also has a direct stimulatory effect on neutrophils. Once all the invaders have been removed, the inflammatory response will subside and the area will be cleared of the remaining “casualties”. Then the process of wound healing starts. Although it is known that neutrophils play a pivotal role in wound healing, it is not clear which neutrophil-derived substances are involved and how the neutrophils are active in healing without being aggressive to the surrounding tissue. In general, damaged tissue will be replaced by scar tissue formed mainly of fibroblasts and collagen.

When inflammation occurs in areas of the body with an important function, like tissues formed from heart muscle cells, brain cells or lung alveolar cells, normal function will be compromised by the resulting scar formation, causing serious conditions like heart failure, paralysis and emphysema. To minimize the debilitating consequences of these conditions, it is important to “dampen” the inflammatory reaction as quickly as possible.

Intervention to control the acute early phase inflammatory response presents an opportunity to improve the prognosis for a wide range of patients whose symptoms can be traced back to such an event. Such an approach has been advocated for many acute and chronic inflammation based diseases and shown to have potential based on findings from relevant disease models. Classical anti-inflammatory drugs such as steroids and Non Steroid Anti-Inflammatory Drugs (NSAIDS) do not have the ideal profile of action, either in terms of efficacy or safety. Steroids affect the “wrong” cell type (monocytes) and their dampening effects are easily bypassed. NSAIDS generally show a relatively mild effect partly because they intervene at a late stage in the inflammatory process. Both classes of drugs produce a range of undesirable side effects resulting from other aspects of their pharmacological activity.

Several drugs under early development only interfere with late mediators in the route to neutrophil activation (e.g. C5 convertase inhibitors, antibodies against C5a, C5a-receptor blocking drugs, antibodies against integrins (like CD11b/CD18) and L-selectin on neutrophils and antibodies against adhesion molecules (like ICAM-1 and E-selectin) on endothelial cells).

Antibodies against TNF and IL-8 have effects in chronic inflammation, but only marginal effects in acute inflammation, because of the minimal role monocytes (which are mainly responsible for these substances' production) play in the acute phase and because they react even later in the inflammation cascade. In many cases it would be extremely desirable to stop the inflammatory cascade in an early-as-possible-phase. This is also true because this cascade is not linear but branches off at different stages causing redundancy in the later steps.

Sometimes, the cause of the acute inflammation cannot be removed and the inflammation becomes chronic. With the exception of tuberculosis, chronic hepatitis and certain other conditions, this is seldom the case with infections. However, chronic inflammation can also be caused by stimuli other than bacteria, such as auto-immune reactions. Research has shown that in chronic inflammation the role of monocytes is much more prominent, and that neutrophil migration and activation, monocyte migration and activation, tissue damage, removal of dead cells and wound healing are all going on at the same time.

This complex cascade of interactions between cells and many different cytokines and chemokines has been the subject of intensive research for many years. It was believed that monocytes and their products were the most important elements that needed to be inhibited to dampen chronic inflammation. This explains why steroids, which specifically interact with monocytes, are generally more effective in chronic as opposed to acute inflammation. Long-term treatment with steroids however, is not a desirable option, because severe and unacceptable side effects can occur at the doses required to produce a clinical effect.

Newer treatments using antibodies against TNF or IL-8 have shown good results, and were initially seen as proof of the major role monocytes were thought to play in chronic inflammation. Recent research casts doubts on an exclusive role for monocytes in inflammation and points to a critical role for neutrophils, which are now seen to represent better targets for therapeutic intervention. Also in chronic inflammation it could still be desirable to dampen the early onset of activation as opposed to totally block one of the later steps. This early step-treatment (e.g. lectin pathway inhibition) could lead in due course to modification of disease progression, or even a complete cure, and not just symptomatic relief.

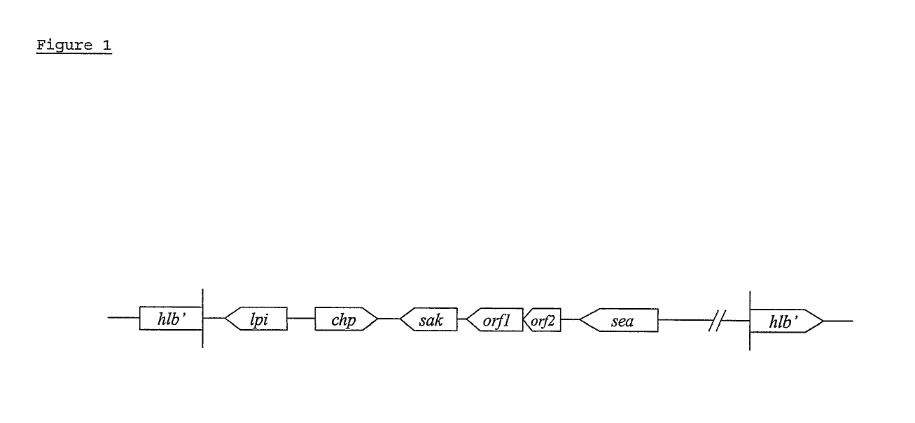

In the research that led to the present invention the gene, the (poly)peptide and its function for a new agent with inflammation-inhibiting properties was found in the bacterium Staphylococcus aureus (S. aureus). Recently, the inventors described a neutrophil modulating agent, CHIPS (Chemotaxis Inhibitory protein of Staphylocci as described in PCT/NL99/00442, which was found to be located on a bacteriophage. On this phage, a so-called Pathogenicity Island was found containing four genes, the genes for CHIPS (chp), Staphylokinase (sak) and enterotoxin A (sea) and a fourth unknown open reading frame (FIG. 1).

The inventors found that the three known genes are virulence factors that have one thing in common. They all interfere with the innate immune system. CHIPS, as they found, inhibits chemotaxis towards C5a and fMLP. Staphylokinase interferes via human plasmin, as the inventors have shown recently, with IgG mediated opsonization and also with complement mediated opsonization. Furthermore, others recently described that SAK causes destruction of defensins, important antimicrobial peptides. Enterotoxin A is described as an superantigen to interfere with adaptive immunity but others also found that it blocks the response to certain chemokines by modulating chemokine receptors.

From this the inventors concluded that the fourth open reading frame within SaPI-5, with unknown function was very likely to encode a protein that would also interfere with innate immunity. The hypothesis was that this open reading frame would inhibit some innate immune mechanism, important to fight staphylococcal infections in one way or another.

To prove this hypothesis the inventors cloned and expressed the protein encoded in this open reading frame in E. coli and purified the protein to homogeneity. This pure protein was evaluated in a number of their in vitro innate immunity assays, in particular: chemotaxis, chemokinesis, cytokine induction (TNF, IL-1, IL-6, IL-10), chemokine induction in whole blood or in isolated monocytes or mononuclear cells, Ca-flux assays with neutrophils or mononuclear cells and flow cytometry, adherence assays (fluorometer), transmigration of neutrophils through endothelium (fluorometric), actin polymerisation (flow cytometrie), phagocytosis (uptake) of standard opsonized erythrocytes (flow cytometry), phagocytosis (opsonization) of standard neutrophils with different opsonins and different bacteria, quantitative bacterial killing assays, membrane depolarization assays (FLEX station), metabolic burst measurements in a luminometer (production O₂-radicals), priming assays for fMLP, PAF etc (luminometer), degranulation assays for MPO and elastase (FLEX station), receptor expression assays (neutrophil/monocyte phenotyping for innate immune receptors, flow cytometry) etc. Hereafter it was concluded that LPI is indeed a modulator of innate immunity. It specifically inhibits the lectin pathway of complement activation. Therefore the protein was named LPI (Lectin Pathway Inhibitor).

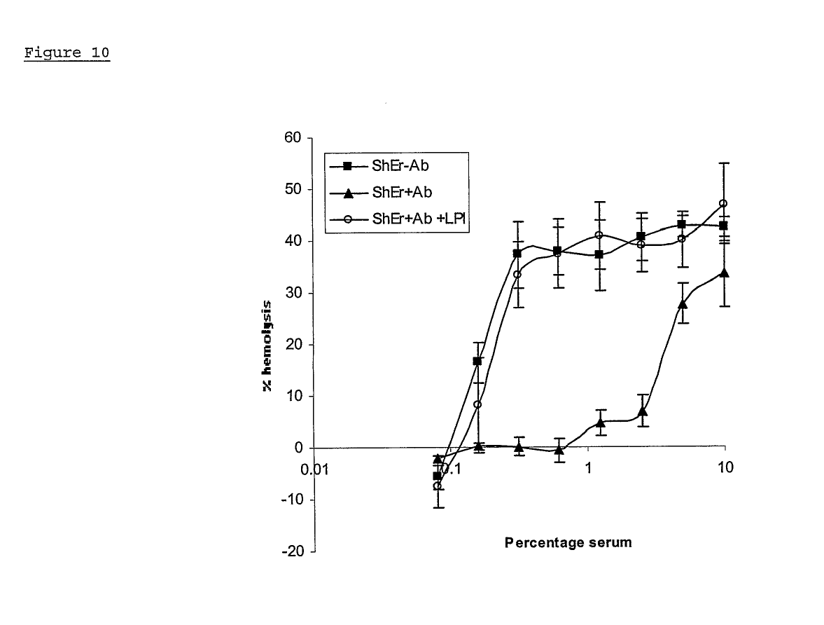

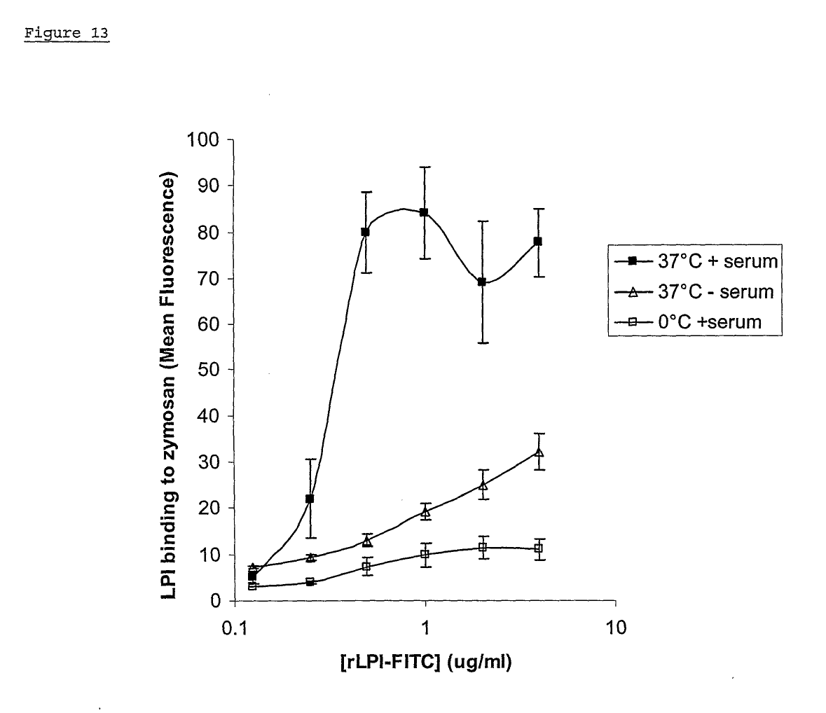

Thus LPI was designed by staphylococcal evolution, specifically to inhibit the lectin pathway of complement activation. This is the pathway, which is the greatest threat to staphylococci in the early phases once they have invaded the human body. Both MBL and ficolins can directly recognize the staphylococcal cell wall and initiate the complement cascade, leading to the attraction of neutrophils (C5a, C3a) and the opsonization of staphylococci. For this reason the inventors also demonstrated the activity of LPI in phagocytosis of staphylococci in Example 3. After evaluating LPI in all separate pathways of the complement cascade the inventors concluded that LPI is a specific lectin pathway inhibitor (FIGS. 10, 11 and 12) After evaluating LPI in all separate steps in the lectin pathway they concluded that LPI inhibits lectin pathway mediated complement activation by inhibiting the C2 cleavage activity of MASP-2 but not the C4 cleavage activity of MASP-2 (FIGS. 13, 14, 15, 16, 17, 18, 19, 20).

Therefore LPI activity is defined as follows: LPI prevents activation of the lectin pathway of complement activation by specifically preventing the MASP-2 dependent cleavage of C2 into C2a and C2b.

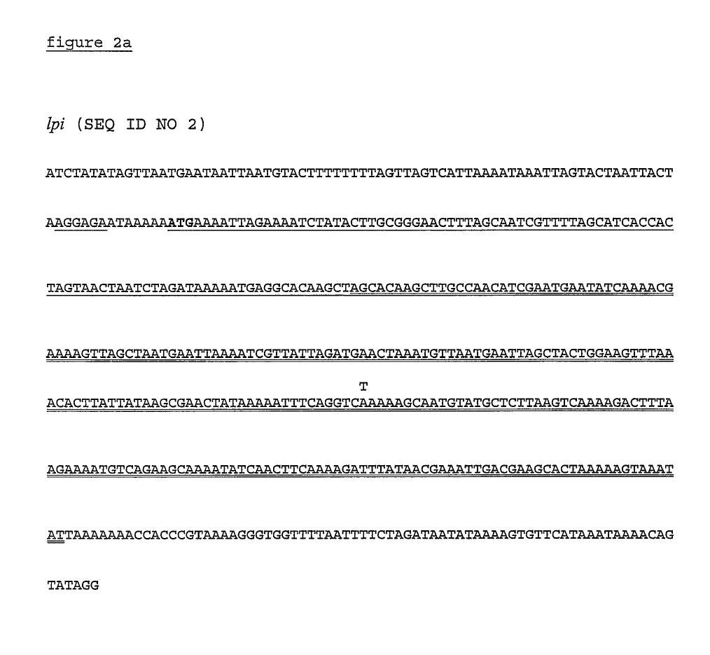

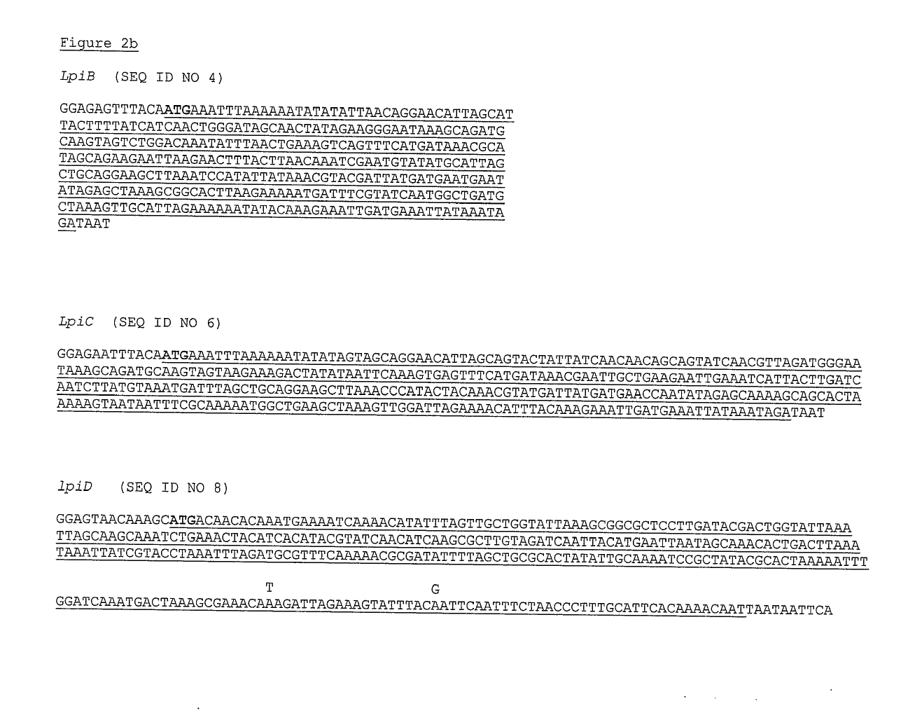

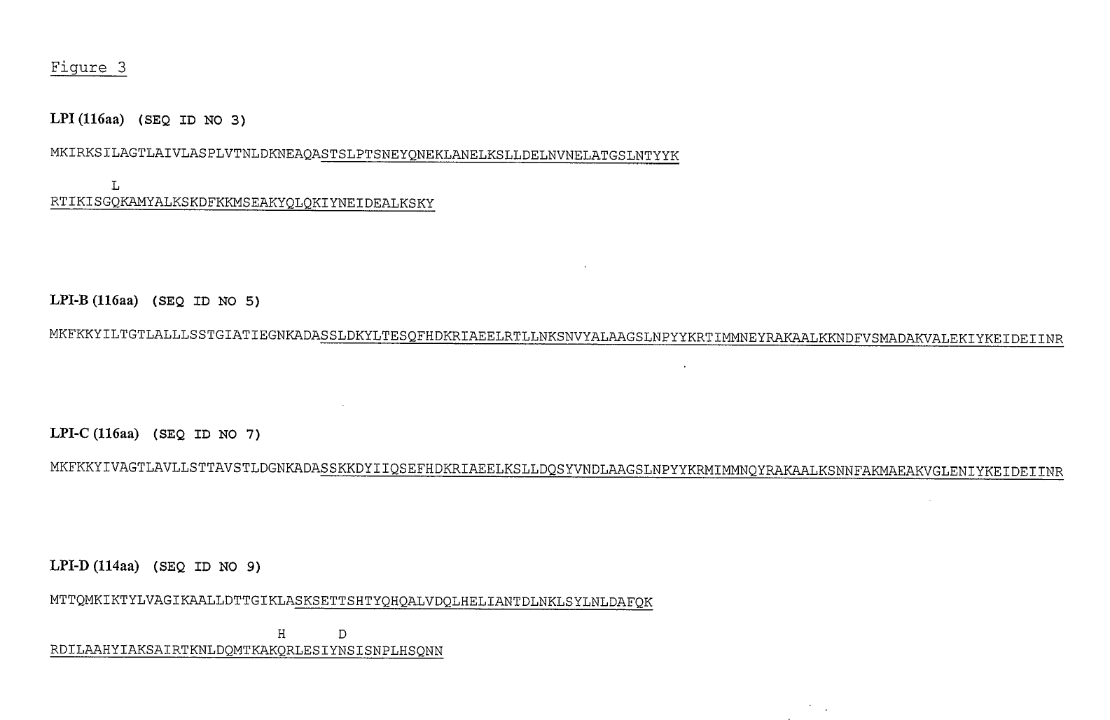

Next to the gene for LPI (FIG. 2a, SEQ ID NO:2) three other homologues genes were identified, also from S. aureus designated lpi-B (SEQ ID NO:4), lpi-C (SEQ ID NO:6) and lpi-D (SEQ ID NO:8). These genes were cloned and expressed and tested for lpi activity. The proteins encoded by these genes are LPI-B (SEQ ID NO:5), nd LPI-C (SEQ ID NO:7) and LPI-D (SEQ ID NO:9) (FIG. 3). It was concluded that LPI-B and LPI-C have LPI-like activity. LPI-D has lower homology and no LPI activity.

The present invention therefore provides a nucleic acid molecule (the gene for LPI is designated lpi) in isolated form, comprising a nucleotide sequence encoding a (poly)peptide having LPI activity, said nucleotide sequence corresponding to a sequence being selected from the group consisting of:

a) a nucleotide sequence comprising at least part of the sequence of lpi, lpi-B or lpi-C as depicted in FIG. 2a or 2b (SEQ ID NO:2, SEQ ID NO:4, SEQ ID NO:6);

b) nucleotide sequences encoding a (poly)peptide having LPI activity and having one of the amino acid sequences depicted in FIG. 3 identified as SEQ ID NO:3, SEQ ID NO:5 or SEQ ID NO:7;

c) nucleotide sequences encoding a (poly)peptide having LPI activity and having a portion of the amino acid sequences depicted in FIG. 3 identified as SEQ ID NO:3, SEQ ID NO:5 or SEQ ID NO:7;

d) nucleotide sequences being at least 40% identical to any one of the nucleotide sequences a), b) or c);

e) nucleotide sequences hybridizing at stringent conditions with any one of the nucleotide sequences a), b), c) or d); and

f) nucleotide sequences complementary to any of the nucleotide sequences a), b), c), d) or e).

The complete genome of Staphylococcus aureus was sequenced and can be found in the regular international databases like GenBank, RefSeq, and PDB. The above sequences identified by SEQ ID NOS 2, 4 and 6 can be found in the database as part of a larger sequence. The nucleic acid in isolated form as related to the described function is however novel. The accession numbers for the hypothetical proteins that are now identified as LPI are gi|14247715|dbj|BAB58104.1|and gi|13701735|dbj|BAB43028.1|. LPI encodes a putative protein of 116 amino acids which shares 35-45% homology with other Staphylococcus aureus proteins of the same size, LPI-B (gi|13700958|dbj|BAB42254.1|; gi|14246929|dbj|BAB57321.1|), and LPI-C (gn1|Sanger_{—}159288|Staphylococcus (from the Sanger database (S. aureus 252, (MRSA 16)).

These sequences are also part of this invention and are depicted in FIG. 2b (lpi-B, SEQ ID NO:4, lpi-C, SEQ ID NO:6).

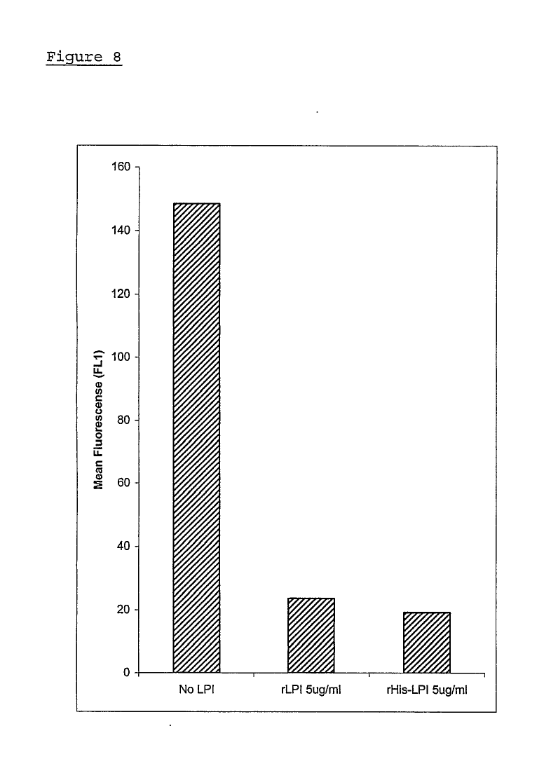

It was shown that the proteins encoded by the homologous genes lpi-B and lpi-C have at least LPI activity (FIG. 8b).

Regarding the term “identical” under d) above it should be noted that identicity and homology are used interchangeably. It should furthermore be noted that for gapped alignments, statistical parameters can be estimated using the Smith-Waterman algorithm that produces optimal alignment scores. Homologues of the LPI nucleic acid sequence or protein sequence are defined by a Gap Open Penalty of at least 12 and a Gap Expression Penalty of at least 1.

The sequence as given in FIG. 2a (SEQ ID NO:2) is one embodiment of the DNA sequence of the invention. It comprises a promoter region from nucleotides 1 to 86, a leader peptide sequence from nucleotides 87 to 179, the coding region for the (poly)peptide having LPI activity from nucleotide 180 to 434, as well as a 3′ untranslated region from nucleotides 435 to 510.

The presented gene for LPI of FIG. 2a or any nucleic acid derived therefrom may for example be operably linked to the trc expression system (Brosius et al., Gene 27: 161-172 (1984)). Many other suitable expression control sequences and methods of expressing recombinant proteins are known (F. M. Ausubel et al., Current Protocols in Molecular Biology, John Wiley and Sons, Inc., New York, N.Y.).

The nucleotide sequence as given in FIG. 2a also contains a leader peptide sequence. The coding region of the mature protein corresponds to nucleotides 180 to 434 of FIG. 2a. Other leader sequences can be used. Or the leader sequence may be omitted entirely, depending on the host cell in which the sequence is to be expressed.

The amino acid sequence in FIG. 3 (LPI) (SEQ ID NO:3) is deduced from the DNA sequence in FIG. 2a. In a further embodiment of the invention the nucleic acid molecule thus may have a nucleotide sequence that corresponds to all degenerate variants of the LPI gene, the lpi-B gene or the lpi-C gene.

The invention furthermore relates to nucleic acid molecules that encode (poly)peptides that do not have the complete sequences LPI (SEQ ID NO:3), LPI-B (SEQ ID NO:5) or LPI-C (SEQ ID NO:7) from FIG. 3 but one or more functional portions thereof that in themselves or together constitute a biologically active (poly)peptide having LPI activity. “A portion” as used herein does not exclude the possibility that a (poly)peptide comprises more than one portion and should thus be interpreted as “at least one”. Such portions may vary in size from the complete amino acid sequence minus one amino acid to peptides of at least 2, preferably at least 5 amino acids. In case the active part of the protein lies in two or more portions of the complete amino acid sequence, the invention also relates to nucleic acid sequences encoding these separate portions in a manner that leads to a peptide configuration that retains the biological activity. In practice this can for example mean that spacer sequences are to be incorporated in between biologically active portions to lead to a biologically active conformation.

Thus, when reference is made to “at least part of the sequence” this means not only the three parts described above (i.e. for LPI: nucleotides 1-434, 87-434 and 180-434) but also other fragments of the gene or combinations thereof provided that they still encode a (poly)peptide having LPI activity.

In a further embodiment thereof, the invention thus provides an isolated nucleic acid molecule of the invention which consists of the coding region of one or more portions of the amino acid sequence LPI (SEQ ID NO:3), LPI-B (SEQ ID NO:5) or LPI-C (SEQ ID NO:7) from FIG. 3, wherein one portion of the amino acid sequence constitutes alone or with other portions of the amino acid sequence the region(s) of the (poly)peptide having LPI activity that lead to biological activity.

The present invention is not limited to nucleic acid molecules having the exact same sequence as the sequence lpi (SEQ ID NO:2), lpi-B (SEQ ID NO:4) or lpi-C (SEQ ID NO:6) depicted in FIGS. 2a and 2b or the above described variants thereof. Therefore, according to the invention additional nucleic acid molecules are provided having a nucleotide sequence which is at least 40%, preferably at least 50%, more preferably at least 60%, even more preferably at least 70%, most preferably at least 80%, the most preferable at least 90% identical or homologous to any one of the nucleotide sequences as defined under a), b) or c) above. Homology is to be determined over the entire length of the homologous sequence.

It was found that LPI is less than 40% homologous to proteins and peptides known to date. Proteins and peptides that show at least 40% amino acid homology to the LPI protein and have LPI activity are thus also part of this invention.

The invention further relates to nucleic acid molecules having a nucleotide sequence hybridizing under stringent conditions with a nucleic acid molecule corresponding with the nucleotide sequence lpi (SEQ ID NO:2), lpi-B (SEQ ID NO:4) or lpi-C (SEQ ID NO:6) given in FIG. 2a or 2b or degenerate sequences thereof, which encode an amino acid sequence LPI (SEQ ID NO:3), LPI-B (SEQ ID NO:5) or LPI-C (SEQ ID NO:7) as given in FIG. 3. Stringent conditions are constituted by overnight hybridization at 42° C. in 5×SSC (SSC=150 mM NaCl, 15 mM trisodium citrate) and washing at 65° C. at 0.1×SSC. In addition to 5×SSC the hybridization solution may comprise 50% formamide, 50 mM sodium phosphate (pH 7.6), 5×Denhardt's solution, 10% dextran sulphate and 20 mg/ml denatured sheared salmon sperm DNA.

The invention is also not limited to the gene which encodes the (poly)peptide having LPI activity, but also relates to nucleic acid molecules that encode fragments, derivatives and analogues thereof. “Fragments” are intended to encompass all parts of the (poly)peptide that retain its biological activity. “Fragments” can consist of one sequence of consecutive amino acids or of more than one of such sequences. “Derivatives” are the complete (poly)peptide having LPI activity or fragments thereof that are modified in some way. Examples of modifications will follow herein below. One example is enabled in Example 3, the His-tagged LPI molecule has also LPI activity. “Analogues” are similar (poly)peptides having LPI activity isolated from other organisms, in particular other pathogenic organisms. All of the above categories have one thing in common, namely that they have “LPI activity”. LPI activity can be measured by any assay that shows inhibition of complement activation. Examples of such assays include deposition of C2, C4 or C3 fragments on a bacterium or erythrocyte, CH50 measurements using lysis of erythrocytes as a readout and others. Therefore, for the present application, the term “(poly)peptides having LPI activity” is intended to include the original LPI, LPI-B and LPI-C proteins and their homologues in isolated or recombinant form, and other (poly)peptides, fragments, derivatives and analogues that exhibit LPI activity.

The isolated nucleic acid molecule according to the invention may be DNA, RNA or cDNA.

The invention furthermore relates to probes and primers derived from the nucleic acid molecule of the invention. Such primers are oligonucleotides or polynucleotides of at least about 10 consecutive nucleotides (nt), and more preferably at least about 25 nt, still more preferably at least about 30 nt, and even more preferably about 30-70 nt of the nucleic acid molecule of the invention. Probes are longer and may for instance be a portion of the nucleic acid molecule of the invention of 50-300 consecutive nt, or even as long as the entire nucleic acid molecule.

Such oligonucleotides or polynucleotides are useful as diagnostic probes or as probes in conventional DNA hybridization techniques or as primers for amplification of a target sequence by polymerase chain reaction (PCR) as described for instance in Ausubel et al. (supra) or other amplification techniques, such as NASBA.

Furthermore, the invention relates to a recombinant vector comprising at least one isolated nucleic acid molecule of the invention. The vector to be used can be selected by the skilled person based on his common general knowledge and will be dependent on the host that is used.

In addition to vectors, the invention provides for a bacteriophage comprising at least one isolated nucleic acid of the invention. In most LPI-positive Staphylococci, the gene encoding LPI is located on a prophage and can be turned into an active phage, for example by treatment with mitomycin-C according to standard and published phage isolating procedures. A bacteriophage is thus a useful vehicle to introduce the LPI gene into a host.

The invention in addition relates to a method for making a recombinant vector, comprising inserting at least one isolated nucleic acid molecule of the invention into a vector. By incorporating more than one copy in the vector, or introducing more than one vector into a host, the level of expression can be influenced. When a host cell is used that comprises an endogenous gene for a corresponding (poly)peptide having LPI activity, the expression level thereof can be increased by introducing more copies of the nucleic acid molecule (i.e. the gene) into the host cell or changing the promoter or regulator regions.

The invention thus also relates to recombinant host cells comprising at least one isolated nucleic acid molecule or vector of the invention. A number of types of organisms or cells from prokaryotes, protists, fungi, animals or plants may act as suitable hosts for the expression of recombinant (poly)peptides having LPI activity. Host cells include the widely used bacterial strain Escherichia coli (E. coli) including, but not limited to, the trc expression system (Brosius et al., supra) that allows high-level, regulated expression from the trc promotor. Potentially suitable othei bacterial strains include Gram-positive bacterial strains, such as Bacillus subtilis, Staphylococcus aureus, or any bacterial strain capable of expressing heterologous proteins. A preferred production process in E. coli is given in Example 2.

The (poly)peptide having LPI activity may also be produced as a recombinant protein using a suitable expression system employing lower eukaryotes such as yeast or insect cells. Suitable yeast strains include Saccharomyces cerevisiae, Pichia pastoris, Candida or any yeast strain capable of expressing heterologous proteins. Insect cells, used for recombinant protein expression include the Drosophila system and the Baculovirus system. Alternatively, it may be possible to produce the (poly)peptide having LPI activity in an mammalian expression system that includes several suitable host cells, including monkey COS, hamster CHO, BHK or RBL-2H3, human 293, 3T3, HeLa, U937, HL-60, or Jurkat cells, mouse L cells and other transformed cells for in vitro culture. For expression of (poly)peptides having LPI activity in eukaryotic systems, it may be necessary to modify the protein produced therein in order to obtain a functional protein. Such modifications, like attachments or substitutions may be accomplished using known chemical or enzymatic methods. In addition, the sequence of the nucleic acid molecule may be adapted to the codon usage of the host cell.

The (poly)peptide having LPI activity of the invention may also be expressed as a product of transgenic animals, e.g. as a component of the milk of transgenic cows, goats, pigs, sheep, rabbits or mice which are characterized by somatic or germ cells containing a nucleotide sequence encoding the (poly)peptide having LPI activity.

Alternatively the (poly)peptide having LPI activity may be expressed in a form that will facilitate purification. For example, it may be tagged with a polyhistidine (6×His) epitope and subsequently purified by using a resin to which nickel ions are bound by means of a chelating agent. The (poly)peptide having LPI activity containing the tag is eluted from the resin by lowering pH or by competing with imidazole or histidine. Such epitope is commercially available from Invitrogen (Breda, The Netherlands). Introduction of a protease cleavage site, like that for enterokinase, enables removal of the fusion tag to generate mature native recombinant (poly)peptide having LPI activity. Materials and methods for such an expression system are commercially available from Invitrogen, using the pTrcHis Xpress™ vectors in combination with ProBound™ resin for efficient isolation of His-tagged protein and EnterokinaseMax™ as highly catalytic active protease and EK-Away™ enterokinase affinity resin to remove the contaminating presence of the protease. Other tags known to those skilled in the art that can be used to facilitate purification include, but are not limited to, glutathion S transferase (GST fusion), myc and HA.

The (poly)peptide having LPI activity may also be produced by known chemical synthesis. Methods for constructing polypeptides or proteins by synthetic means are known to those skilled in the art. The synthetic protein, by virtue of sharing primary, secondary and tertiary structural and/or conformational characteristics with the corresponding (poly)peptide having LPI activity will posses an activity in common therewith, meaning LPI properties. Thus, such synthetically produced proteins can be employed as biologically active or immunological substitute for natural purified (poly)peptide having LPI activity.

The (poly)peptides having LPI activity provided herein also include (poly)peptides characterized by amino acid sequences into which modifications are naturally provided or deliberately engineered. Modifications in the (poly)peptide or DNA sequences can be made by those skilled in the art using known conventional techniques. Modifications of interest in the LPI active (poly)peptide sequences may include replacement, insertion or deletion of selected amino acid residues in the coding sequence.

The information contained in the LPI protein, its gene and other (poly)peptides having LPI activity and their encoding nucleic acid molecules derived therefrom can be used to screen for fragments thereof or other agents which are capable of inhibiting or blocking binding of a (poly)peptide having LPI activity in complement activation assays, and thus may act as inhibitors of LPI binding to its putative target. Appropriate screening assays may for example use the fluorescent labelled purified LPI protein that binds to bacteria in the presence of an intact complement system and analysed by flow cytometry or fluorometry. A suitable binding assay may alternatively employ purified LPI-target or target-domain on a carrier with a form of LPI protein as binder. Alternatively, an assay can be employed that screens for the ability to bind or compete with LPI for binding to a specific anti-LPI antibody (monoclonal, polyclonal, or single chain antibody) by various immunoassays known in the art, including but not limited to competitive and non-competitive ELISA techniques or Biosensor technology employing a sensor chip coated with either ligand (LPI), antibody or putative LPI target (Surface Plasma Resonance (SPR) technique like the BiaCore). Any (poly)peptide having LPI activity other than LPI may also be used in the screening assays described. All these methods can be adapted for High Throughput Screening (HTS).

The functional activity of LPI, the (poly)peptides, their fragments, derivatives and analogues can be assayed by various methods. Al methods that measure complement activation at one of its steps can be used as a readout because LPI interferes with the first steps in this process. Thus, C4, C2, or C3 fragments-deposition (by flow cytometry or ELISA or immunoblotting), CH50 measurements using erythrocyte lysis, measurement of MAC complex or soluble split products of the complement cascade (ELISA or functional assays) are all suitable candidates for measuring LPI activity. Examples 3, 4, 5, 7 and 8 describe such methods.

Isolated (poly)peptides having LPI activity may be useful in treating, preventing or ameliorating inflammatory conditions that are involved in many diseases and disorders, such as those listed in Table 2.

According to a further aspect thereof, the invention thus relates to (poly)peptides having LPI activity for use in diagnosis, prophylaxis or therapy, in particular for use in the treatment of acute and chronic inflammation reactions, such as those listed in Table 2.

TABLE 2
Diseases caused by inflammatory reactions, involving
complement activation and/or neutrophil and or monocyte
involvement.

acute reactive arthritis
acute transplant rejection
adult respiratory distress
syndrome (ARDS)
alcoholic hepatitis
allergic rhinitis
allotransplantation
Alzheimer's disease
arteriosclerosis
arthus reaction
asthma
atherosclerosis
atopic dermatitis
bacterial meningitis
bacterial pneumonia
brain tumour
bronchogenic carcinoma
bullos pemphigoid
burn injuries
burns
cardiopulmonary bypass
cardiovascular diseases
chronic bronchitis
chronic lymph leukemia
chronic obstructive pulmonary
disease (COPD)
contact dermatitis
Crohn's disease
cutaneous T-cell lymphoma
cystic fibrosis
dermatoses
diseases of the central nervous
system
endometriosis
experimental allergic
encephalomyelitis (EAE)
experimental allergic neuritis
(EAN)
Forssman shock
frost bite
gastric carcinoma
gastrointestinal diseases
genitourinary diseases
glomerulonephritis
gout
haemolytic anemia
Heliobacter pylori gastritis
hemodialysis
hereditary angioedema
hypersensitivity pneumonia
idiopathic pulmonary fibrosis
immune complex (IC)-induced
vasculitis
ischaemic shock
ischaemia-reperfusion episodes
ischemia-reperfusion injuries
joint diseases
(large) vessel surgery
metal fume fever
multiple sclerosis
multiple system organ failure
myasthenia gravis
Mycobacterium tuberculosis
infection
myocardial infarction
pancreatitis
peritonitis
pleural emphesema
post-cardiopulmonary bypass
(CBP) inflammation
psoriasis
repetitive strain injury (RSI)
respiratory diseases
rheumatoid arthritis
sepsis
septic shock
sinusitis
skin diseases
stroke
systemic lupus erythematosus
(SLE)
transplantation
(traumatic) brain injury
Trichomonas vaginalis infection
ulcerative colitis
urinary tract infection
vascular leak syndrome
vasculitis
viral hepatitis
viral meningitis
viral respiratory tract
infection
xenotransplantation
*Support for the therapeutical usefulness of the (poly)peptides of the invention for treatment of these diseases can be found in the following references: For ARDS: Demling RH (1995). The modern version of adult respiratory distress syndrome. Ann. Rev. Med. 46: 193-202; and Fujishima S, Aikawa N 1995 Neutrophil mediated tissue injury and its modulation. Intensive Care Med 21: 277-285; For severe infections (meningitis): Tunkel AR and Scheld WM (1993). Pathogenesis and pathophysiology of bacterial meningitis. Clin. Microbiol. Rev. 6: 118. For injury after ischaemia/reperfusion: Helier T, et al. (1999). Selection of a C5a receptor antagonist from phage libraries attenuating the inflammatory response in immune complex disease and ischemia/reperfusion injury. J. Immunol. 163: 985-994. For rheumatoid arthritis: Edwards SW and Hallett MB (1997). Seeing the wood for the trees: the forgotten role of neutrophils in rheumatoid arthritis. Immunology Today 18: 320-324; and Pillinger MH, Abramson SB (1995). The neutrophil in rheumatoid arthritis. Rheum. Dis. Clin. North Am. 1995 21: 691-714. For myocardial infarction: Byrne JG, Smith WJ, Murphy MP, Couper GS, Appleyard RF, Cohn LH (1992). Complete prevention of myocardial stunning, contracture, low reflow, and edema after heart transplantation by blocking neutrophil adhesion molecules during reperfusion. J. Thorac. Cardiovasc. Surg. 104: 1589-96. For COPD: Cox G (1998). The role of neutrophils in inflammation. Can. Respir. J. 5 Suppl A: 37A-40A; and Hiemstra PS, van Wetering S, Stolk J (1998). Neutrophil serine proteinases and defensins in chronic obstructive pulmonary disease: effects on pulmonary epithelium. Eur. Respir. J. 12: 1200-1208. For stroke: Barone FC, Feuerstein GZ (1999). Inflammatory mediators and stroke: new opportunities for novel therapeutics. J. Cereb. Blood Flow Metab. 19: 819-834; and Jean WC, Spellman SR, Nussbaum ES, Low WC (1998). Reperfusion injury after focal cerebral ischemia: the role of inflammation and the therapeutic horizon. Neurosurgery 43: 1382-1396. For meningitis: Tuomanen EI (1996). Molecular and cellular mechanisms of pneumococcal meningitis. Ann. N. Y. Acad. Sci. 797: 42 52. For all directly complement related diseases: Adapted from: A. Sahu and J. D. Lambris Immunopharmacology 49 (2000) 133-148.

The invention furthermore relates to the use of the (poly)peptides having LPI activity for the manufacture of a preparation for diagnosis, prophylaxis or therapy, in particular for the treatment of acute and chronic inflammation reactions, more in particular for the treatment of the indications referred to above.

Also part of the present invention are therapeutic compositions comprising a suitable excipient and one or more of the (poly)peptide having LPI activity of the invention. Such composition can be used for the treatments as specified above.

The invention further relates to use of the nucleic acid molecule of the invention, optionally incorporated in a larger construct, for various purposes, such as raising antibodies thereto, modulating the LPI activity or in a therapeutic preparation.

The invention further relates to nucleic acid molecules and the amino acid sequence encoded by the nucleic acid molecules that can be identified by so-called “computer cloning”. More specifically, this technique comprises using:

(1) the nucleic acid sequence lpi (SEQ ID NO:2), lpi-B (SEQ ID NO:4) or lpi-C (SEQ ID NO:6) as depicted in FIG. 2, or fragments, derivatives and analogues thereof, or

(2) the amino acid sequence LPI (SEQ ID NO:3), LPI-B (SEQ ID NO:5) or LPI-C (SEQ ID NO:7) as depicted in FIG. 3, or fragments, derivatives and analogues thereof, as a query. for screening nucleic acid sequences or nucleic acid sequence databases, or protein sequences or protein sequence databases, using search algorithms that can identify regions with homology. Such algorithms are known to the person skilled in the art and include, but are not limited to, BLAST searches (Altschul et al., J. Mol. Biol. 215, 403-410 (1990)). The sequence databases that may be searched include, but are not limited to, the Genbank™ database and the Swissprot™ database. When using a BLAST search or modifications thereof, generally subjects that display homology can be identified. Identification is based on the value of the Score or the Smallest Sum Probability P(N). Homologues of the LPI nucleic acid sequence or (poly)peptide sequence are defined by a Score that is at least 200, preferably at least 400, more preferably at least 800, most preferably at least 1600. Alternatively, the P(N) value can be used for identification of homologous sequences. Homologues of the LPI nucleic acid sequence or (poly)peptide sequence are defined by a P(N) value that is smaller than 1e-3, preferably smaller than 1e-6, more preferably smaller than 1e-12, even more preferably smaller than 1e-24, most preferably smaller than 1e-48.

The isolated nucleic acid molecules of the invention can furthermore be used for gene therapy. The nucleic acid molecule can be introduced at the site of inflammation to act locally or at a distant site. Gene therapy is via viral vectors, such as, but not limited to, adenoviral vectors, adeno-associated viral vectors or lentiviral vectors. Alternatively, non-viral vectors, such as those based on liposomes or polymers may be used. Gene therapeutic strategies are based on (1) in vivo gene therapy, where the isolated nucleic acid molecules of the invention are introduced into target cells in vivo, or (2) ex vivo gene therapy, where the isolated nucleic acid molecules of the invention are introduced into target cells ex vivo, followed by administration of the transduced cells, or a sub-population of the transduced cells, into an individual. The invention also relates to the vectors for use in gene therapy and to transduced cells.

The invention relates to a method for treating a subject suffering from inflammation by administering a therapeutically effective amount of a (poly)peptide of the invention and a method for gene therapeutically treating a subject suffering from inflammation by administering a therapeutically effective amount of a nucleic acid molecule, as well as a method for treating a subject suffering from staphylococcus infection by administering a therapeutically effective amount of an antibody and/or biologically active fragment thereof.

The nucleic acid molecules of the invention can be used in a method for isolating from an organism a gene encoding a protein having LPI activity, which method comprises screening of a genomic or cDNA library of that organism with a probe based on the nucleic acid molecule, and isolation of the positive clones.

According to a further aspect thereof, the invention relates to micro-organisms harboring one or more nucleic acid molecules of the invention for use as a medicament for the treatment of acute and chronic inflammation reactions, such as listed in Table 2.

All molecules of the invention, i.e. nucleic acid molecules, (poly)peptides, non-(poly)peptides, fragments, derivatives and analogues, may find various other applications. Such applications include, but are not limited to:
Isolation of factors that can bind the above mentioned molecules. Examples of such factors being receptors and proteins. Such isolation can for instance be performed using the yeast two hybrid system or using tagged molecules of the invention as bait for fishing.
Making phage display libraries, which can in turn be used for determining active domains, functional equivalents etc.
Identifying signal transduction pathways that are activated or inactivated by LPI and the molecules of the invention.
Assay for determination of the biological LPI activity (receptor expression upregulation).

According to this invention peptoids and peptidomimetics can be designed on the basis of the claimed LPI (poly)peptides.

Various definitions for peptidomimetics have been formulated in literature. Among others, peptidomimetics have been described as “chemical structures designed to convert the information contained in peptides into small non-peptide structures”, “molecules that mimic the biological activity of peptides but no longer contain any peptide bonds”, “structures which serve as appropriate substitutes for peptides in interactions with receptors and enzymes” and as “chemical Trojan horses”.

In general, peptidomimetics can be classified into two categories. The first consists of compounds with non-peptide-like structures, often scaffolds onto which pharmacophoric groups have been attached. Thus, they are low molecular-weight compounds and bear no structural resemblance to the native peptides, resulting in an increased stability towards proteolytic enzymes.

The second main class of peptidomimetics consists of compounds of a modular construction comparable to that of (poly)peptides. These compounds can be obtained by modification of either the (poly)peptide side chains or the (poly)peptide backbone. Peptidomimetics of the latter category can be considered to be derived of (poly)peptides by replacement of the amide bond with other moieties. As a result, the compounds are expected to be less sensitive to degradation by proteases. Modification of the amide bond also influences other characteristics such as lipophilicity, hydrogen bonding capacity and conformational flexibility, which in favourable cases may result in an overall improved pharmacological and/or pharmaceutical profile of the compound.

Oligomeric peptidomimetics can in principle be prepared starting from monomeric building blocks in repeating cycles of reaction steps. Therefore, these compounds may be suitable for automated synthesis analogous to the well-established preparation of peptides in peptide synthesizers. Another application of the monomeric building blocks lies in the preparation of peptide/peptidomimetic hybrids, combining natural amino acids and peptidomimetic building blocks to give products in which only some of the amide bonds have been replaced. This may result in compounds which differ sufficiently from the native peptide to obtain an increased biostability, but still possess enough resemblance to the original structure to retain the biological activity.

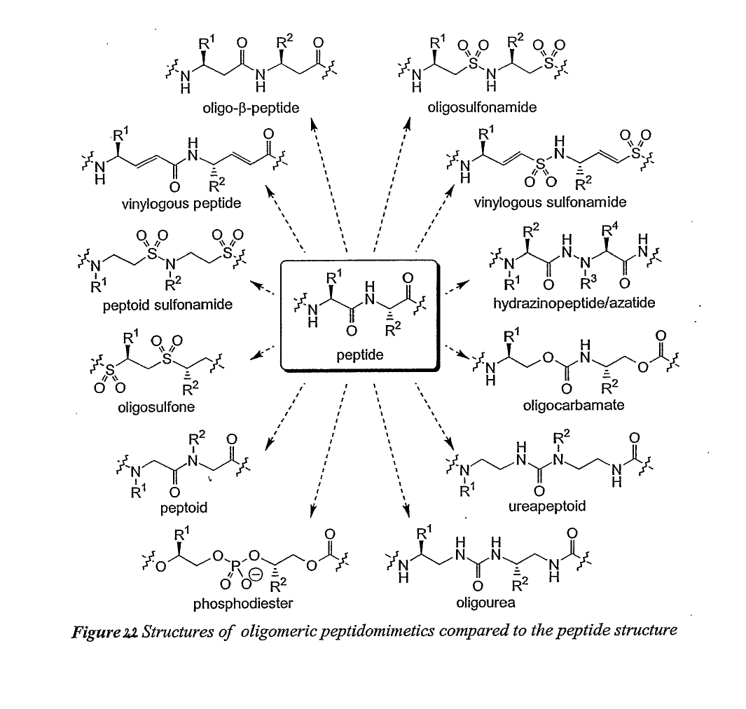

Suitable peptidomimetic building blocks for use in the invention are amide bond surrogates, such as the oligo-5-peptides (Juaristi, E. Enantioselective Synthesis of b-Amino Acids; Wiley-VCH: New York, 1996), vinylogous peptides (Hagihari, M. et al., J. Am. Chem. Soc. 1992, 114, 10672-10674), peptoids (Simon, R. J. et al., Proc. Natl. Acad. Sci. USA 1992, 89, 9367-9371; Zuckermann, R. N. et al., J. Med. Chem. 1994, 37, 2678-2685; Kruijtzer, J. A. W. & Liskamp, R. M. J. Tetrahedron Lett. 1995, 36, 6969-6972); Kruijtzer, J. A. W. Thesis; Utrecht University, 1996; Kruijtzer, J. A. W. et al., Chem. Eur. J. 1998, 4, 1570-1580), oligosulfones (Sommerfield, T. & Seebach, D. Angew. Chem., Int. Ed. Eng. 1995, 34, 553-554), phosphodiesters (Lin, P. S.; Ganesan, A. Bioorg. Med. Chem. Lett. 1998, 8, 511-514), oligosulfonamides (Moree, W. J. et al., Tetrahedron Lett. 1991, 32, 409-412; Moree, W. J. et al., Tetrahedron Lett. 1992, 33, 6389-6392; Moree, W. J. et al., Tetrahedron 1993, 49, 1133-1150; Moree, W. J. Thesis; Leiden University, 1994; Moree, W. J. et al., J. Org. Chem. 1995, 60, 5157-5169; de Bont, D. B. A. et al., Bioorg. Med. Chem. Lett. 1996, 6, 3035-3040; de Bont, D. B. A. et al., Bioorg. Med. Chem. 1996, 4, 667-672; Lowik, D. W. P. M. Thesis; Utrecht University, 1998), peptoid sulfonamides (van Ameijde, J. & Liskamp, R. M. J. Tetrahedron Lett. 2000, 41, 1103-1106), vinylogous sulfonamides (Gennari, C. et al., Eur. J. Org. Chem. 1998, 2437-2449), azatides (or hydrazinopeptides) (Han, H. & Janda, K. D. J. Am. Chem. Soc. 1996, 118, 2539-2544), oligocarbamates (Paikoff, S. J. et al., Tetrahedron Lett. 1996, 37, 5653-5656; Cho, C. Y. et al., Science 1993, 261, 1303-1305), ureapeptoids (Kruijtzer, J. A. W. et al., Tetrahedron Lett. 1997, 38, 5335-5338; Wilson, M. E. & Nowick, J. S. Tetrahedron Lett. 1998, 39, 6613-6616) and oligopyrrolinones (Smith III, A. B. et al., J. Am. Chem. Soc. 1992, 114, 10672-10674). FIG. 22 shows the structures of these peptidomimetic building blocks.

The vinylogous peptides and oligopyrrolinones have been developed in order to be able to form secondary structures (β-strand conformations) similar to those of peptides, or mimic secondary structures of peptides. All these oligomeric peptidomimetics are expected to be resistant to proteases and can be assembled in high-yielding coupling reactions from optically active monomers (except the peptoids).

Peptidosulfonamides are composed of α- or β-substituted amino ethane sulfonamides containing one or more sulfonamide transition-state isosteres, as an analog of the hydrolysis of the amide bond. Peptide analogs containing a transition-state analog of the hydrolysis of the amide bond have found a widespread use in the development of protease inhibitor e.g. HIV-protease inhibitors.

Another approach to develop oligomeric peptidomimetics is to completely modify the peptide backbone by replacement of all amide bonds by non-hydrolyzable surrogates e.g. carbamate, sulfone, urea and sulfonamide groups. Such oligomeric peptidomimetics may have an increased metabolic stability. Recently, an amide-based alternative oligomeric peptidomimetics has been designed viz. N-substituted Glycine-oligopeptides, the so-called peptoids. Peptoids are characterized by the presence of the amino acid side chain on the amide nitrogen as opposed to being present on the α-C-atom in a peptide, which leads to an increased metabolic stability, as well as removal of the backbone chirality. The absence of the chiral α-C atom can be considered as an advantage because spatial restrictions which are present in peptides do not exist when dealing with peptoids. Furthermore, the space between the side chain and the carbonyl group in a peptoid is identical to that in a peptide. Despite the differences between peptides and peptoids, they have been shown to give rise to biologically active compounds.

Translation of a (poly)peptide chain into a peptoid peptidomimetic may result in either a peptoid (direct-translation) or a retropeptoid (retro-sequence). In the latter category the relative orientation of the carbonyl groups to the side chains is maintained leading to a better resemblance to the parent peptide.

Review articles about peptidomimetics that are incorporated herein by reference are: Adang, A. E. P. et al.; Recl. Tray. Chim. Pays-Bas 1994, 113, 63-78; Giannis, A. & Kolter, T. Angew. Chem. Int. Ed. Engl. 1993, 32, 1244-1267; Moos, W. H. et al., Annu. Rep. Med. Chem. 1993, 28, 315-324; Gallop, M. A. et al., J. Med. Chem. 1994, 37, 1233-1251; Olson, G. L. et al., J. Med. Chem. 1993, 36, 3039-30304; Liskamp, R. M. J. Recl. Tray. Chim. Pays-Bas 1994, 113, 1-19; Liskamp, R. M. J. Angew. Chem. Int. Ed. Engl. 1994, 33, 305-307; Gante, J. Angew. Chem. Int. Ed. Engl. 1994, 33, 1699-1720; Gordon, E. M. et al., Med. Chem. 1994, 37, 1385-1401; and Liskamp, R. M. J. Angew. Chem. Int. Ed. Engl. 1994, 33, 633-636.

The invention thus furthermore relates to molecules that are not LPI (poly)peptides themselves but have a structure and function similar to those of the LPI (poly)peptides described herein. Examples of such molecules are the above described peptidomimetics, but also compounds in which one or more of the amino acids are replaced by non-proteinogenic amino acids or D-amino acids. When reference is made in this application to (poly)peptides, it is intended to include also such other compounds that have a similar or the same structure and function and as a consequence a similar or the same biological LPI activity as the (poly) peptides.

More in particular substitutions can be made with non-proteinogenic amino acids selected from the group consisting of 2-naphtylalanine (NaI(2)), β-cyclohexylalanine (Cha), p-amino-phenylalanine ((Phe(p-NH₂), p-benzoyl-phenylalanine (Bpa), ornithine (Orn), norleucine (Nle), 4-fluoro-phenylalanine (Phe(p-F)), 4-chloro-phenylalanine (Phe(p-Cl)), 4-bromo-phenylalanine (Phe(p-Br)), 4-iodo-phenylalanine (Phe(p-I)), 4-methyl-phenylalanine (Phe(p-Me)), 4-methoxy-phenylalanine (Tyr(Me)), 4-nitro-phenylalanine (Phe(p-NO2)).

Suitable D-amino acids for substituting the amino acids in the (poly)peptides of the invention are for example those that are selected from the group consisting of D-phenylalanine, D-alanine, D-arginine, D-asparagine, D-aspartic acid, D-cysteine, D-glutamic acid, D-glutamine, D-histidine, D-isoleucine, D-leucine, D-lysine, D-methionine, D-proline, D-serine, D-threonine, D-tryptophan, D-tyrosine, D-valine, D-2-naphtylalanine (D-NaI(2)), β-cyclohexyl-D-alanine (D-Cha), 4-amino-D-phenylalanine (D-Phe(p-NH₂)), p-benzoyl-D-phenylalanine (D-Bpa), D-Ornithine (D-Orn), D-Norleucine (D-Nle), 4-fluoro-D-phenylalanine (D-Phe(p-F)), 4-chloro-D-phenylalanine (D-Phe(p-Cl)), 4-bromo-D-phenylalanine (D-Phe(p-Br)), 4-iodo-D-phenylalanine (D-Phe(p-I)), 4-methyl-D-phenylalanine (D-Phe(p-Me)), 4-methoxy-D-phenylalanine (D-Tyr(Me)), 4-nitro-D-phenylalanine (D-Phe(p-NO2)).

One or more of the amino acids in the (poly)peptides can be replaced by peptoid building blocks, e.g. selected from the group consisting of N-substituted glycines, such as N-benzylglycine (NPhe), N-methylglycine (NAla), N-(3-guanidinopropyl)glycine (NArg), N—(Carboxymethyl)glycine (NAsp), N-(carbamylmethyl)glycine (NAsn), N-(thioethyl)-glycine (NhCys), N-(2-carboxyethyl)glycine (NGlu), N-(2-carbamylethyl)glycine (NGln), N-(imidazolylethyl)glycine (NhHis), N-(1-methylpropyl)glycine (Nile), N-(2-methylpropyl)glycine (NLeu), N-(4-aminobutyl)glycine (NLys), N-(2-methylthioethyl)glycine (NMet), N-(hydroxyethyl)glycine (NhSer), N-(2-hydroxypropyl)glycine (NhThr), N-(3-indolylmethyl)glycine (NTrp), N-(p-hydroxyphenmethyl)-glycine (NTyr), N-(1-methylethyl)glycine (NVal).

All compounds of the invention may also be in cyclic form. A cyclic compound may have improved potency, stability, rigidity and/or other pharmaceutical and/or pharmacological characteristics.

All molecules of the invention can be labelled in any way. Examples of labelling include but are not limited to fluorescence, biotin, radioactive labelling etc. Such labelled molecules can be used for screening of compounds that resemble or overlap with the biological activity of LPI, as well as identification of binding sites, both in vivo and in vitro, and for tracing LPI protein or nucleic acid in an organism.

The present invention will be further illustrated in the examples that follow and that are in no way intended to be limiting to this invention. In this description and the examples reference is made to the following figures and tables:

FIG. 1 illustrates the organization of the genes in the part of the bacteriophage that was called the pathogenicity Island SaPI-5. Shown is SaPI-5 in the 5′ region of the bacteriophage of MRSA-16 which is incorporated in the structural gene of β-toxin. The position and orientation of the four genes of interest: lpi, chp, sak and sea are indicated. Orf1 and orf2 represent structural genes of the bacteriophage.

FIG. 2a shows the sequence of the LPI gene from S. aureus MRSA-16. The Shine Dalgarno sequence (AGGAGA) and the LPI open reading frame (ORF) are underlined. The nucleotides encoding the mature protein are indicated by a double line. The diverging nucleotide of S. aureus NCTC 8325 and N315 is indicated above the sequence.

FIG. 2b shows the sequence of the LPI-B, LPI-C and LPI-D genes. The LPI-B and LPI-C open reading frames are underlined. The diverging nucleotides in different LPI-D sequences is indicated above the sequence.

FIG. 3 shows the amino acid sequence deduced for the LPI, LPI-B, LPI-C and LPI-D genes. The region matching the mature LPI, LPI-B, LPI-C and LPI-D protein is underlined. The diverging amino acids in LPI and LPI-D are indicated above the sequence.

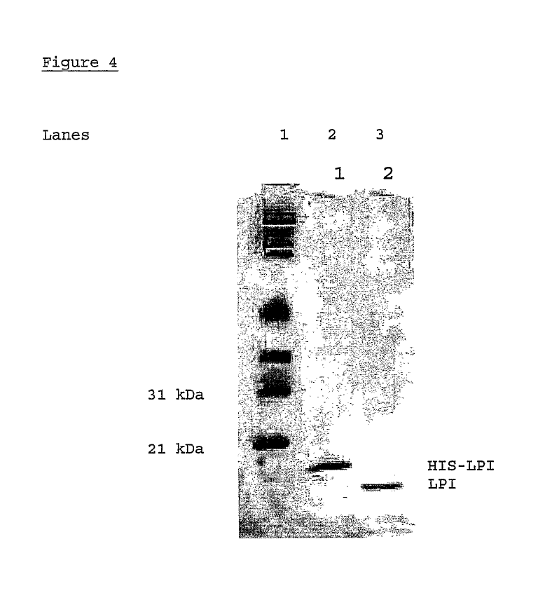

FIG. 4 is a representative image of an SDS-PAGE showing the final purified recombinant LPI (rLPI) obtained from an E. coli lysate after affinity chromatography over a Nickel column and cleavage of the Histidine tag by Enterokinase.

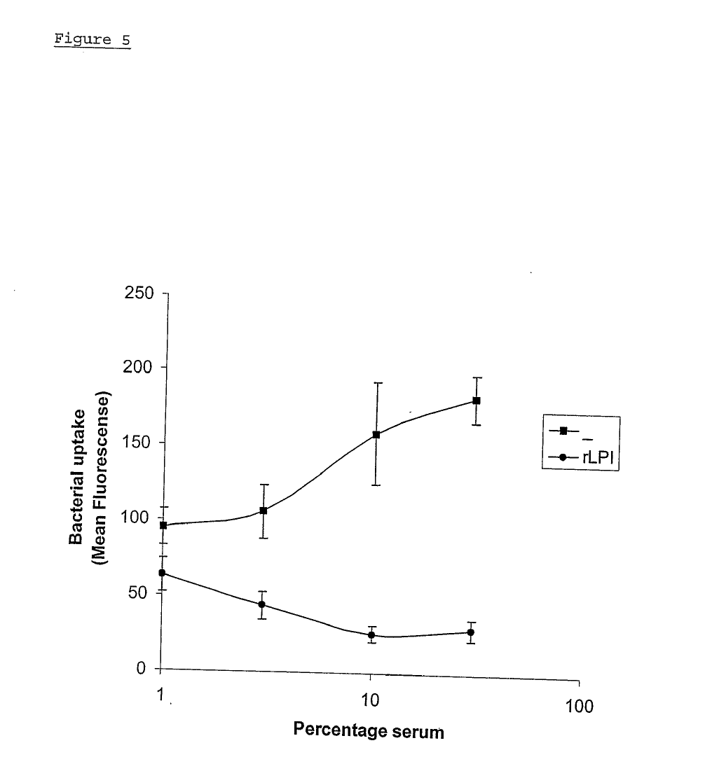

FIG. 5 shows the effects of rLPI treatment on the uptake of S. aureus by human neutrophils. Labelled bacteria were incubated with human sera or isolated immunoglobulins and neutrophils in the presence or absence of rLPI for 15 minutes. Phagocytosis was measured by flow cytometry and FIG. 5 shows the inhibitory effect of 3 μg/ml rLPI on the uptake of S. aureus in human sera.

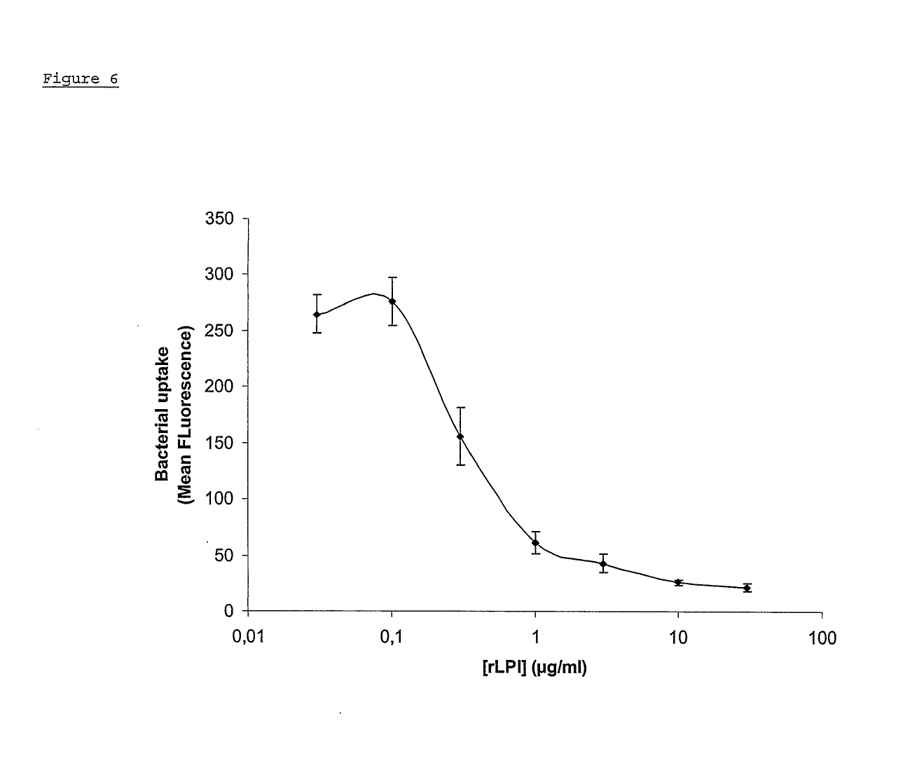

FIG. 6 illustrates the dose-dependent inhibitory effect of rLPI on bacterial uptake. Phagocytosis was performed in 10% human sera.

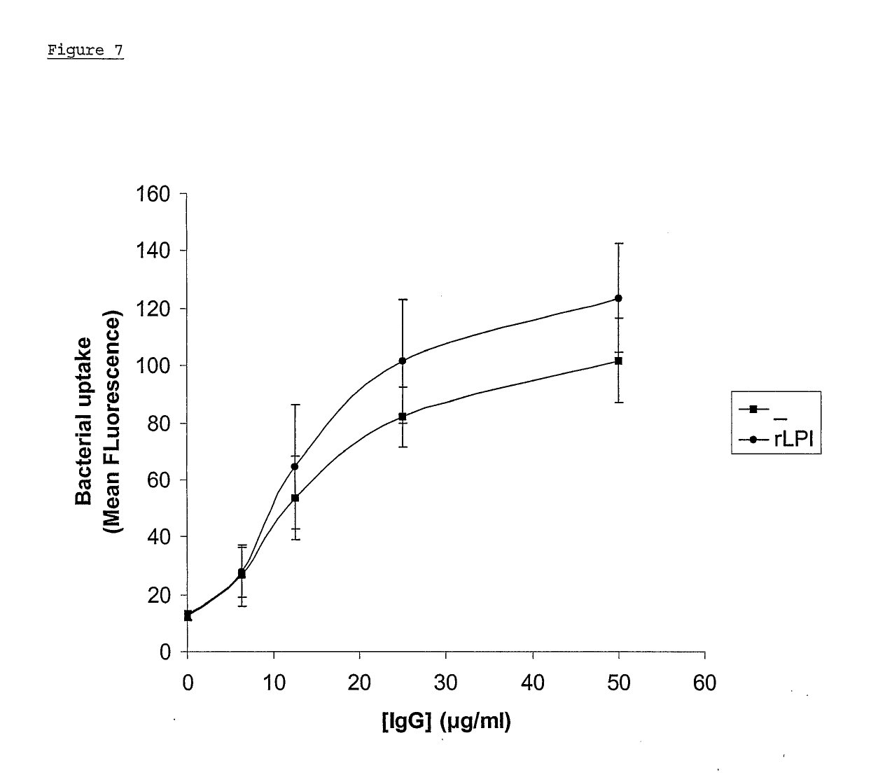

In FIG. 7 the lack of effect is illustrated of 8 rLPI on the Fcγ-receptors mediated phagocytosis of S. aureus by neutrophils, tested by incubating bacteria and neutrophils with purified human immunoglobulins only, thus in the absence of complement.

FIG. 8 shows that there are other molecules then LPI that have LPI activity. His-tagged LPI has the same activity as normal rLPI.

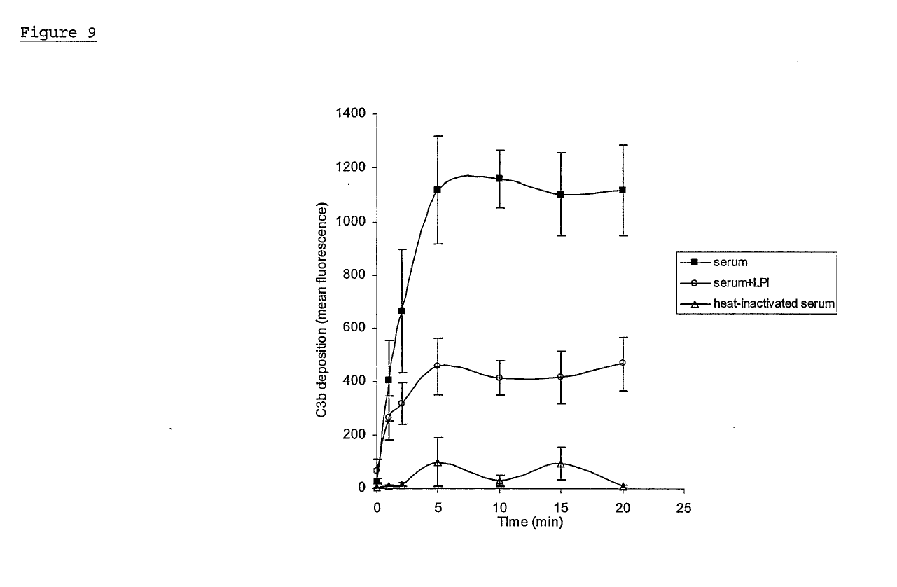

FIG. 9 represents the inhibitory effect of rLPI on the deposition of C3b molecules on the surface of S. aureus. C3b deposition was performed by incubation of S. aureus in 10% human serum in the presence of rLPI in time. Bacteria were washed and the amount of C3b on the surface was detected by specific antibodies labelled with fluorescein. C3b deposition was measured by flow cytometry.

FIG. 10 shows the lack of inhibitory effect of rLPI on classical pathway activation as measured in a haemolysis assay.

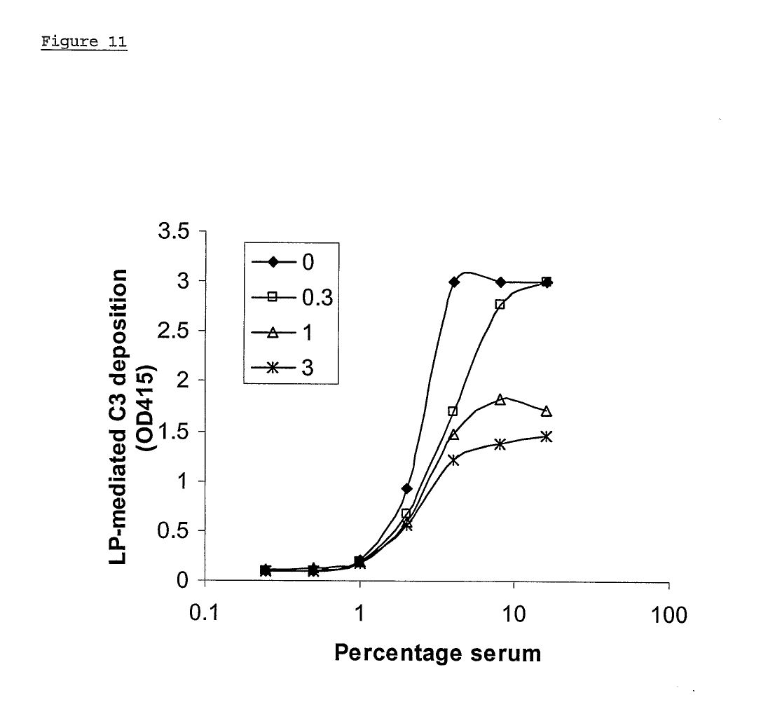

FIG. 11 shows the inhibitory effect of rLPI on lectin pathway activation. An ELISA based method on mannan-coated plates with C1q depleted serum and C3b deposition as a read-out were used.

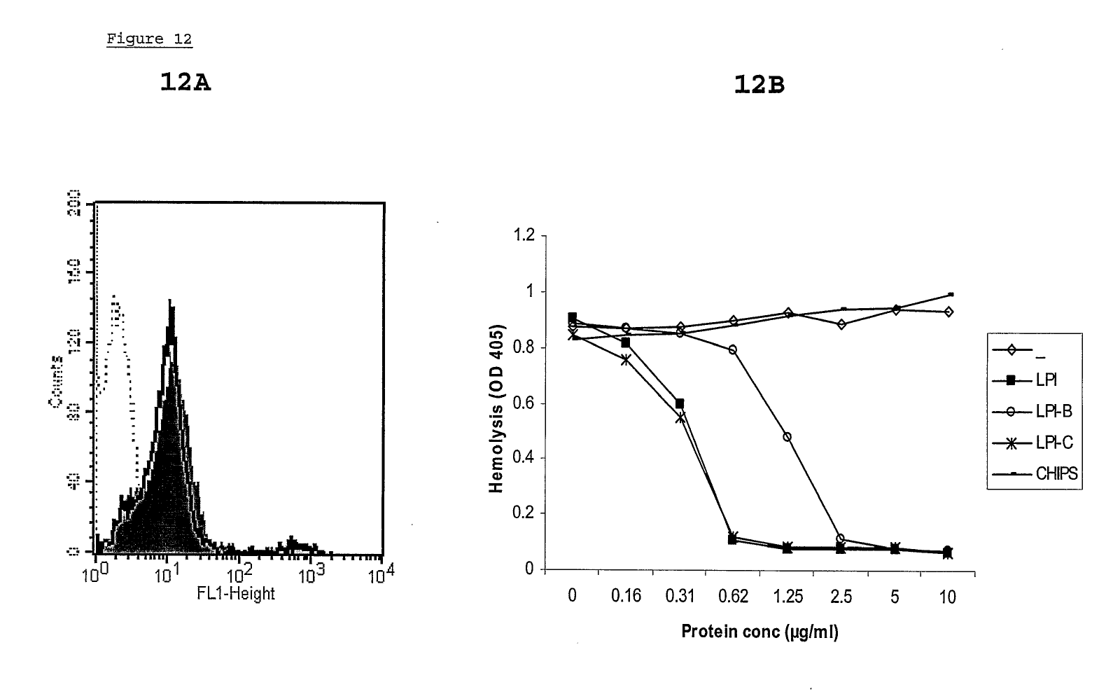

FIG. 12 shows the effect of rLPI on alternative pathway activation. In FIG. 12A the alternative pathway activation was assessed by flow cytometric analysis of deposition of C3b on zymosan using only purified C3, factor D, factor B and properdin (factor P) FIG. 12B shows the activity of LPI, LPI-B and LPI-C in an alternative pathway assay, measured in a hemolytic assay using rabbit erythrocytes.

FIG. 13 depicts the binding of LPI to zymosan particles. rLPI was labelled with fluorescein and incubated with zymosan in the presence of human serum at various temperatures. Then fluorescence per particle was measured using flow cytometry.

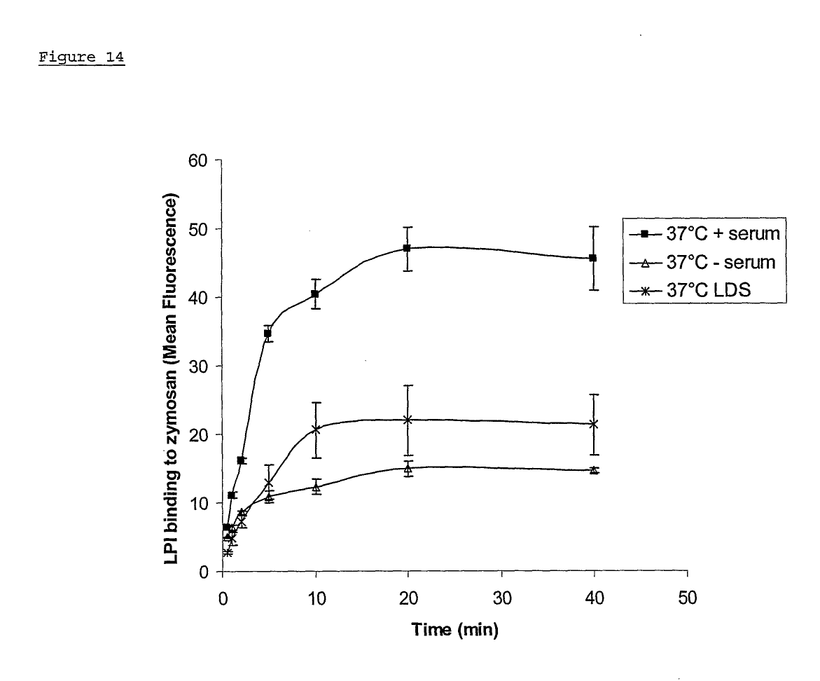

FIG. 14 depicts the binding of LPI to zymosan particles. rLPI was labelled with fluorescein and incubated with zymosan with and without serum or with Lectin depleted serum (LDS) at various time points. Then fluorescence per particle was measured using flow cytometry.

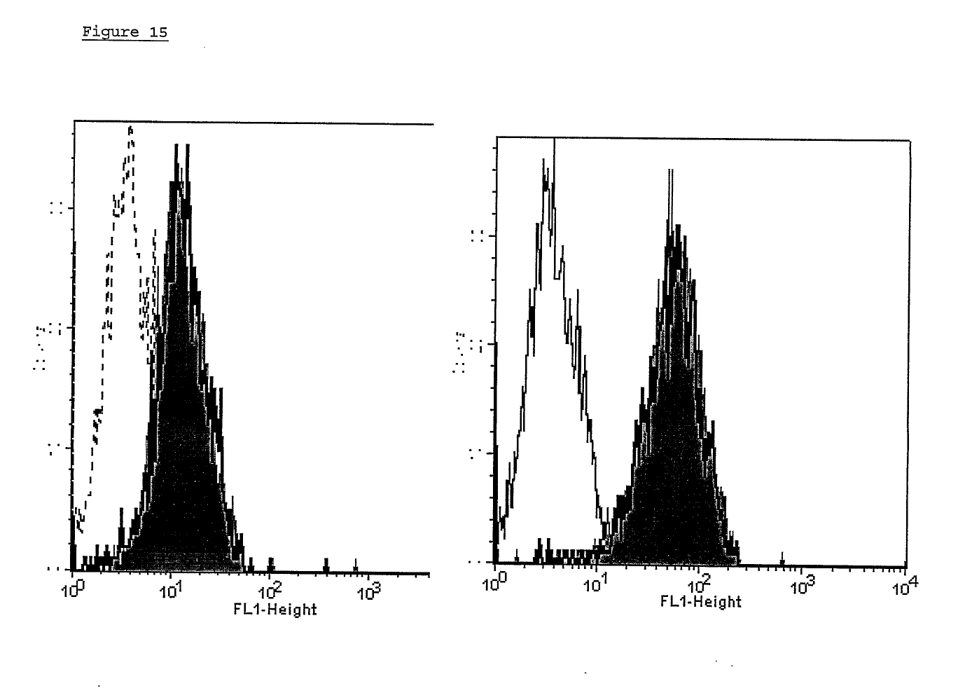

FIG. 15 Depicts the binding of MBL to zymosan particles in the presence and absence of LPI. MBL was detected by flow cytometry after staining with a specific anti-MBL antibody.

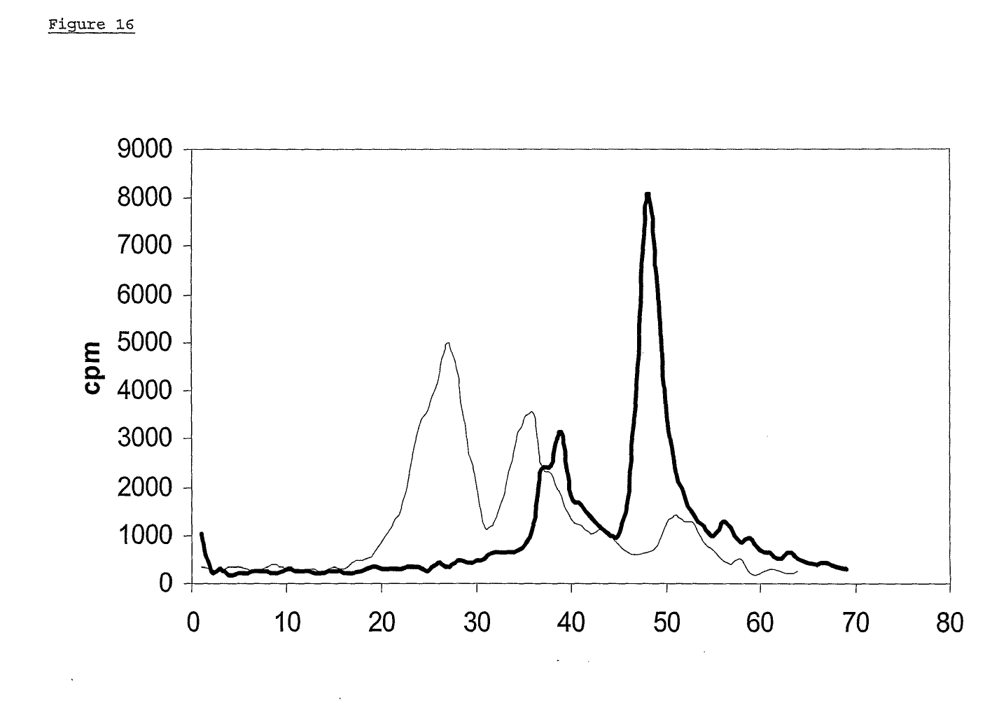

FIG. 16 shows the association between of rLPI and recombinant human MASP-2 as analyzed by size-exclusion chromatography using iodinated rLPI.

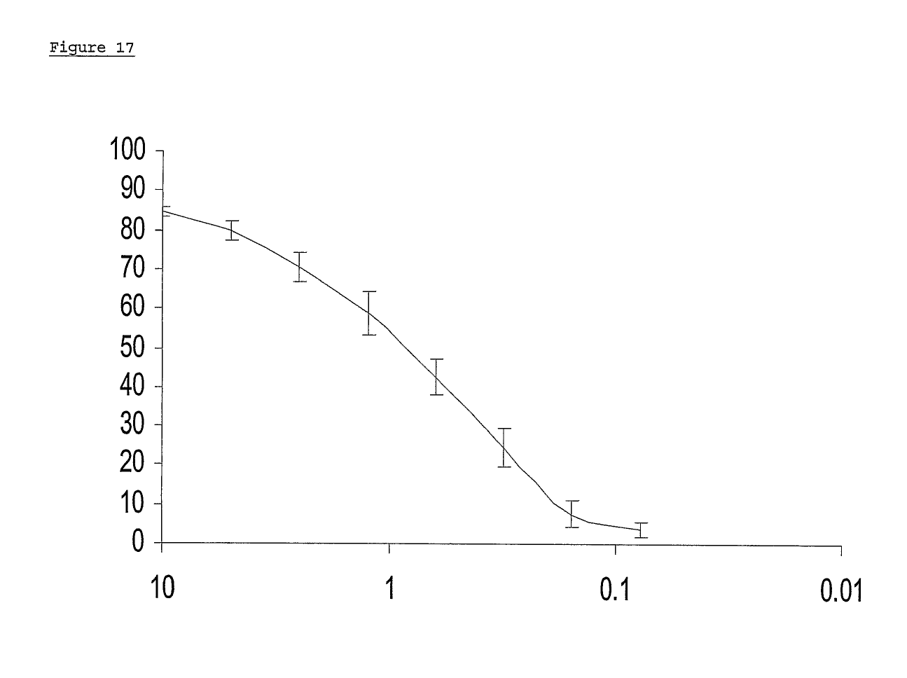

FIG. 17 shows the inhibition of rMASP-2 in a specific proteolytic cleavage assay, using a fluorescent substrate by rLPI.

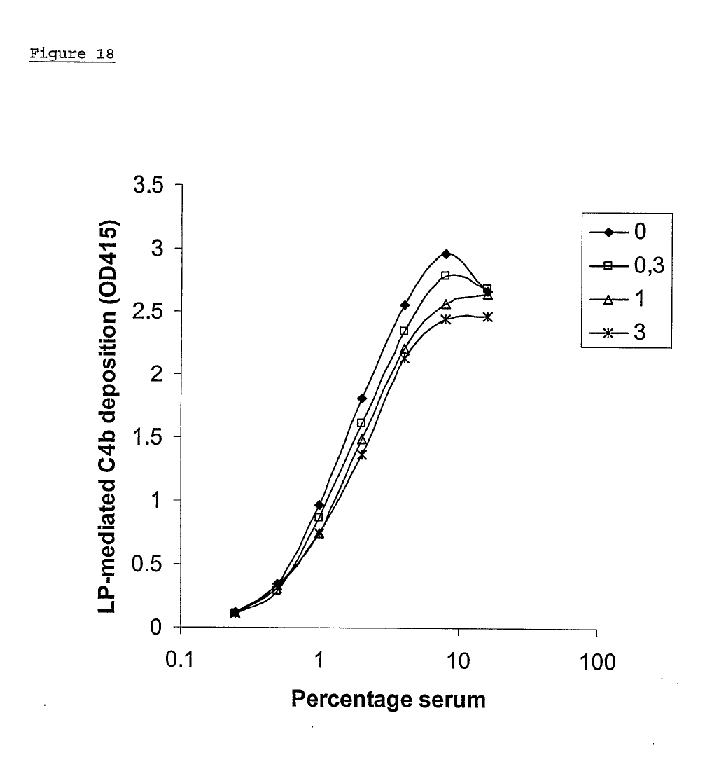

FIG. 18 shows Lectin Pathway-mediated C4b deposition on mannan-coated plates in different amounts of serum and the lack of inhibition by increasing amounts of rLPI.

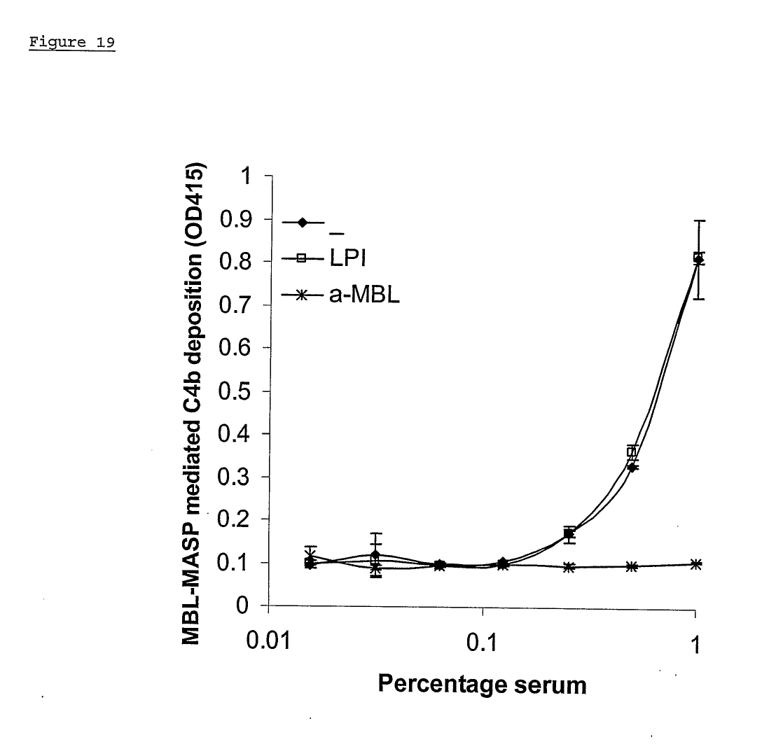

FIG. 19 shows C4b deposition as a result of cleavage of purified C4 by MBL-MASPs captured on mannan coated plates and the lack of inhibition by LPI compared to that of an anti-MBL antibody (total inhibition).

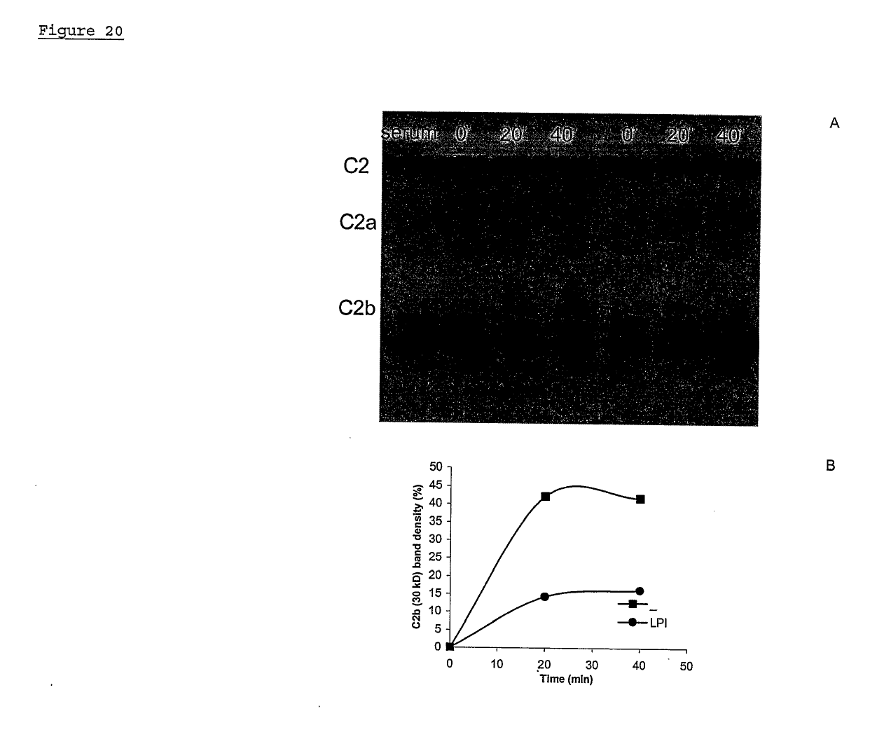

FIG. 20 shows the inhibitory effect of rLPI on C2 cleavage into C2a and C2b as analysed by Western blotting in serum at various time periods FIG. 20a shows the blot after staining with anti-C2. FIG. 20b shows the densitometric analysis of this same blot for the specific C2b band.

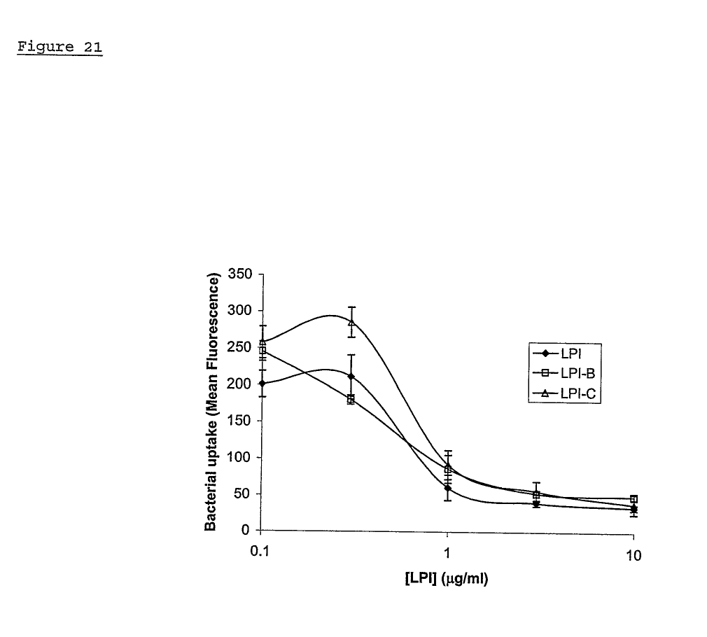

FIG. 21 shows the activity of related LPI molecules, LPI-B and LPI-C, in a phagocytosis assay similar to FIG. 5.

FIG. 22 shows peptidomimetic building blocks.

### EXAMPLES

### Example 1

### Identification of LPI as an Immunomodulating Protein of S. aureus

### 1.1 Introducing Staphylococcus aureus Pathogenicity Island-5 (SaPI-5)

Recently the inventors' lab described CHIPS, a Chemotaxis Inhibitory Protein of Staphylococcus aureus. From data obtained of different S. aureus genome sequence projects it became clear that MRSA 16, NCTC 8325, and N315 carry the gene for CHIPS (chp). In each strain chp is located on a different bacteriophage inserted in the structural gene for β-hemolysin (hlb). The chp carrying bacteriophage of MRSA-16 (phi-MRSA-16), bacteriophages of NCTC 8325 (phi-NCTC 8325) and the bacteriophages of N315 (phi-N315) are approximately 45 kb and have almost identical 5′ regions. The remaining 37 kb of phi-MRSA-16, phi-NCTC 8325 and phi-N315 are poorly homologous.

Beside chp the 5′ homologous region of the three bacteriophages can contain the genes for the virulence factors: staphylokinase (sak), enterotoxin A (sea) (In N315 sea is replaced by enterotoxin P (sep)). Sak is located just downstream of chp, sea or sep (in N315) is positioned just upstream of sak (FIG. 1). In addition, MSSA and Mu50 carry the bacteriophages phi-MSSA and phi-Mu50 containing a 5′ end almost identical to phi-MRSA 16, phi-NCTC 8325, and phi-N315. On the 5′ end of phi-MSSA and phi-Mu50, sak and sea were found in the same conformation as the three other bacteriophages, except for a 0.78 kb chp cassette that is missing. The remaining 32.1 kb of this phage share little similarity to phi-MRSA-16, phi-NCTC 8325 phi-N315 or with each other.

In strain NU3-1 a 4.2 kb fragment was found carrying both chp and sak identical to the 5′ region of phi-MRSA-16, phi-NCTC 8325, phi-N315, phi-MSSA and phi-Mu50 yet in this case no sign of a bacteriophage was found (T. Horii et al., FEMS Microbial Letters 185:221-224 (2000)). So the chp and sak-encoding region of NU3-1 is a direct insertion in the genome of S. aureus. Based on these data it is believed that chp, sak and sea are located on Staphylococcus aureus pathogenicity island-5 (SaPI-5). SaPI-5 can contain up to 3 known virulence factors in a strict order (FIG. 1).

### 1.2 SaPI-5 a Cluster of Staphylococcal Immune Modulatory Proteins

CHIPS interacts specifically with the C5a receptor (C5aR) as well as the formylated peptide receptor (FPR) of human neutrophils resulting in the specific and total downregulation of the response to both receptors. Staphylokinase (SAK) is described as a thrombolytic factor, by transforming plasminogen into the protease plasmin (M. Parry et al., TIBS 25:53-59 (2000)). The inventors recently found that SAK and plasmin locate on the staphylococcal surface and are there capable of cleaving human IgG at the hinge region, thereby destroying its opsonic features. In addition to that, surface located plasmin also cleaved C3b, again removing opsonic molecules from the surface of staphylococci. Thus also SAK has strong anti-innate immunity and thus anti-inflammatory properties.

Enterotoxins are well known to be superantigenic (M. Dinges et al., Clin. Microbial. Rev., 13:16-34 (2000)) but recently Enterotoxin A is also described to down-regulate the receptors CCR1, CCR2 and CCR5 on human peripheral blood monocytes resulting in decreased chemokine responsiveness of these cells (R. Rahimpour et al., J. of Imm. 162:2299-2307 1999)).

As mentioned above chp, sak and sea or sep are all located on SaPI-5, which means we are dealing here with a cluster of genes playing an important role in innate immune modulation.

### 1.3 LPI is Part of SaPI-5

Beside these three known immune modulators an open reading frame was found on SaPI-5 encoding a putative protein of 116 amino. Analyses revealed we are dealing with an excreted protein since it contains a classical staphylococcus aureus signal peptide. Because of its location on SaPI-5 next to three Staphylococcal immune modulators we speculated on a similar function of this protein.

### Results

### SaPI-5

FIG. 1 shows the organization of SaPI-5 in the 5′ region of phi-MRSA-16, which is incorporated in the structural gene of β-toxin. The position and orientation of LPI, chp, sak and sea are indicated. Orf1 and orf2 represent structural genes of the bacteriophage.

### LPI (Lectin Pathway Inhibitor)

The gene was named lpi. It encodes an open reading frame of 348 by preceded by a reasonable Shine Dalgarno sequence for initiation of translation (J. Shine and L. Dalgarno, Proc. Natl. Acad. Sci. USA, 71:1342-1346 (1974)) and followed by one stop codon (FIG. 2a). In FIG. 2a, the Shine Dalgarno sequence (AGGAGA) and the LPI open reading frame (ORF) are underlined. The nucleotides encoding the mature protein are indicated by a double line. The diverging nucleotide of S. aureus NCTC 8325 and N315 is indicated above the sequence. The sequences of homologous genes lpi-B and lpi-C that are also part of this invention are given in FIG. 2b together with the sequence for lpi-D. The LPI-B, LPI-C and LPI-D open reading frames are underlined. Besides these three genes no further significant homology was found with other genes or proteins in the databases to date. The N-terminal 31 amino acids seem to form a signal peptide for secretion across the cytoplasmic membrane (3 positively charged residues followed by a non-charged region of 20 amino acids and an ALA-X-ALA consensus motive for cleavage by the signal peptidase 1 (FIG. 3) (G. von Heijne, Nucl. Acids Res. 14:4683-4690 (1986)).

FIG. 3 shows the amino acid sequence deduced for the LPI, LPI-B, LPI-C and LPI-D genes. The region matching the mature LPI, LPI-B, LPI-C or LPI-D protein is underlined. The deduced mature protein LPI has a size of 85 amino acids and 9.8 kDa and an isoelectric point of 9.06 LPI was found in five different S. aureus stains. In Mu50 the protein was named BAB58104 and in N315 BAB43028. The sequences were compared and found to be identical with one exception. In LPI of NCTC 8325 and N315 adenine on position 315 is replaced by a thymidine, which leads to amino acid sequence change glutamine on position 80 into leucine (FIGS. 2a and 3). In FIG. 3 also the sequences of LPI-B, LPI-C and LPI-D are shown. Mature proteins have the following characteristics:

LPI-B: 85 amino acids and 9.9 kDa and an isoelectric point of 9.18
LPI-C: 85 amino acids and 9.9 kDa and an isoelectric point of 8.88
LPI-D: 86 amino acids and 9.9 kDa and an isolelectric point of 9.06.

### Example 2

### Cloning, and Expression of the LPI-, LPI-B- and LPI-C-Encoding Genes (lpi, lpi-B and lpi-C) of Staphylococcus aureus

### Material and Method

### 3.1 Bacterial Strains, Plasmids and Growth Conditions

Staphylococcus aureus Newman was used as the source of LPI, Escherichia coli DH5α was used as a cloning host (F. M. Ausubel et al., Current Protocols in Molecular Biology, John Wiley and Sons, Inc., New York, N.Y. (1990)). All strains were grown in BM broth (1% tryptone, 0.5% yeast extract, 0.5% NaCl, 0.1% K₂HPO₄, 0.1% glucose) at 37° C. unless otherwise noted.

### 3.2 Production of Recombinant Polypeptides Having LPI Activity in E. coli

The production method used to produced LPI in E. coli can also be used for other (poly)peptides having LPI activity. This production method is illustrated herein below.

The DNA sequence for LPI, LPI-B or LPI-C from S. aureus is cloned into a suitable vector that enables efficient expression of LPI in competent E. coli host cells using conventional molecular biology techniques. The strategy used enables expression of the complete LPI or LPI-like protein linked to a removable HIS-tag at the N-terminus in the cytoplasm of E. coli. The T7 Expression System (pRSET B vector; Invitrogen) was used that enables expression of non-toxic proteins in E. coli. This system uses the strong phage T7 promotor for high-level, regulated expression in any E. coli strain with a multicloning vector. The vector contains an N-terminal polyhistidine (6×His) tag for rapid purification, an Xpress epitope for easy detection with an anti-Xpress antibody and an Enterokinase cleavage site for removal of fusion tag. The Enterokinase recognition site of the pRSET-B vector was changed from GAC GAT GAC GAT AAG into GAC GAT GAC GAC AAG so that blunt end digestion by PshAl (Westburg BV, Leusden, The Netherlands) was possible. An extra guanine at the 5′ end of the LPI primers was used to complement the enterokinase cleavage site.

S. aureus Newman chromosomal DNA was used as template for the PCR reaction using the PfuTurbo DNA polymerase (Stratagene) that results in a blunt ended PCR product. The primers used are LPI-5′ (gagcacaagcttgccaacatcg) (an extra guanine followed with exactly the first amino acid of LPI) and LPI-3′ (ccggaattcttaatatttactttttagtgc) (containing the autologous stop codon and a EcoRI-site).

The same procedure was performed for the LPI-B gene from S. aureus Newman chromosomal DNA. This fragment is 255 bp, the primers used are lpi-B-5′ (gagtagtctggacaaatattt) and lpi-B-3′ (ccggaattcttatctatttataatttcat).

The same procedure was performed for the LPI-C gene from chromosomal DNA of an S. aureus clinical isolate, positive for lpi-C. This fragment is 255 bp, the primers used are lpi-C-5′ (GAGTAGTAAGAAAGACTATAT) and lpi-C-3′ (GGAATTCCTTATCTATTTATAATTTCA).

The PCR product is digested with EcoRI and the pRSET B vector with PshAl to create a blunt end. Thereafter the vector is digested with EcoRI and ligated with the digested PCR product.

For transformation of the vector, Rosetta-gami(DE3) pLysS E. coli competent cells (Novagen) were used. Clones are screened on carbenicillin, chloramphenicol, kanamycine and tetracycline (Sigma) containing plates and proper ligation of lpi is verified by sequencing of the isolated plasmid.

After expression of the LPI, LPI-B or LPI-C gene, the E. coli bacteria are lysed and the protein mixture is applied onto His-Trap resin columns (Amersham Biosciences). For that a culture in LB medium is initiated with 1 mM IPTG for 2 h at 37° C. Bacteria are centrifuged and the pellet resuspended in guanidine lysis buffer (6 M guanidine hydrochloride, 20 mM sodium phosphate, 500 mM sodium chloride; pH 7.8), incubated at room temperature for 15 min with rocking, and was then sonicated three times at high intensity for 5 s on ice. After removal of the insoluble debris by centrifugation, lysates were applied to a pre-equilibrated His-Trap resin column.

The column was loaded with 0.1 M nickel sulfate solution and equilibrated with denaturing binding buffer (8 M ureum, 20 mM sodium phosphate, 500 mM sodium chloride; pH 7.8). After application of the lysate, the column was washed with ureum binding buffer with decreasing pH (pH 7.8, pH 6.0 and pH 5.3). Column-bound proteins were brought into native phosphate buffer by changing ureum binding buffer (pH 5.3) into native buffer (200 mM sodium dihydrogen phosphate dihydrate, 5 M sodium chloride, 50 mM sodium phosphate dibasic; pH 5.3) solution. Finally, proteins were eluted in 0.05 M EDTA.

The HIS-tag is removed by enterokinase cleavage followed by removal of the protease with an EK-Away enterokinase preparation. Therefor the eluate is dialyzed overnight in cold digestion buffer (50 mM Tris-HCl, 1 mM CaCl2 and 0.1% Tween-20, pH 8.0), filtered through a 0.45 μm filter and digested with 0.175 μl Enterokinase/ml HIS-LPI product. This amount of Enterokinase is batch-dependent and results in a partial digestion to avoid the generation of breakdown products. The digested product is dialyzed against phosphate buffer pH 7.8 and passed over a fresh Nickel column to eliminate His tags and uncleaved His-tagged LPI (HIS-LPI); the run through is pure recombinant LPI (rLPI). Undigested HIS-LPI can be eluted again from Nickel column for a second digestion round. The Nickel column is finally washed with 50 mM EDTA, 0.5 M NaOH, water, 5 mg/ml NiCl₂, water and stored in 20% ethanol.

All steps in the isolation and digestion of HIS-LPI are checked by SDS-PAGE on a 16.5% Tris-Tricine Ready gel (BioRad). Samples are mixed 1:1 with sample buffer (200 mM Tris-HCl pH 6.8, 2% SDS, 40% glycerol, 0.04% Coomassie), boiled for 5 min and loaded on the gel.

The HIS-tag of the expressed protein contains an X-press epitope that enables detection of the HIS-LPI product by Western blot using the anti-X-press antibody (Invitrogen). Proteins are transferred to a nitrocellulose membrane, blocked with 4% gelatin in PBS and probed with the antibody and the appropriate secondary peroxidase labelled conjugate (Harlow & Lane, 1988, Antibodies: a laboratory manual, Cold Spring Harbor Laboratory). The exact same procedure was followed for His-tagged LPI-B and LPI-C.

### Results

FIG. 4 is a representative image of an SDS-PAGE showing the purified recombinant LPI (rLPI). The first lane (1) shows the complete recombinant product that is encoded by the vector generating the LPI protein with an additional Histidine tag and enterokinase cleavage site. This encodes for a protein with an apparent molecular weight of 13 kDa, while purified enterokinase treated LPI (lane 2) runs at an apparent molecular weight of 10 kDa. Preparations of HIS-tagged and cleaved LPI-B and LPI-C were equally pure.

### Example 3

Phagocytosis is an important immunological process that is performed by human phagocytes to eliminate invading bacteria. The following sequential steps can be discriminated, opsonization/recognition/binding, then ingestion of the bacterium by the phagocyte and finally killing and degradation of the bacterium and its toxic compounds.

### 3.1 Phagocytosis

Phagocytosis is a result of recognition of a foreign particle by specific receptors on the plasma membrane of phagocytes. Neutrophils express receptors for serum-derived opsonins, including IgG and opsonic fragments of the complement component C3 (C3b and C3bi). In this example phagocytosis of S. aureus by human neutrophils in the presence of human serum was assayed. Moreover, human IgG fractions were isolated from healthy donors and pure IgG molecules used to study Fcγ-receptor mediated (=IgG-mediated) phagocytosis. Recombinant LPI was added to these assays to gain insight in the role of LPI as an immuno-modulating molecule.

### Materials and Methods

An overnight culture of bacteria was grown an additional 4 hours in fresh medium. Bacteria were washed and incubated for 1 hour at 37° C. with 100 μg/ml FITC in 0.1 M carbonate buffer, pH9.6. Free FITC was removed by washing bacteria 2 times. Human neutrophils were purified on a Ficoll/Histopaque gradient as described previously (Troelstra et al., J. Leukocyte Biol. 61, 173-178 (1997)) using heparinized whole blood from a single donor. Human sera were obtained by pooling sera of 10 healthy donors. A total of 50 μl (1.3×10⁷ cfu/ml) of labelled bacteria were incubated with 100 μl human serum, 50 rLPI (various concentrations) and 50 μl of 1×10⁶ neutrophils.

Bacteria were mixed with neutrophils in a ratio of 10:1. Phagocytosis was allowed for 15 minutes at 37° C. and stopped by fixing samples in 100 ml of 1% paraformaldehyde. Samples were evaluated by flow cytometry.

### Results

FIG. 5 shows the inhibitory effect of 3 μg/ml rLPI on the uptake of S. aureus in normal human serum. Labelled bacteria were incubated with increasing concentrations of human serum in the presence or absence of rLPI. Phagocytosis by human neutrophils was depicted as the mean bacterial uptake by neutrophils. The uptake of labelled bacteria by human neutrophils increases with higher serum concentrations. This figure shows a strong inhibitory effect of rLPI on bacterial uptake.

FIG. 6 illustrates the dose-dependent inhibitory effect of rLPI on bacterial uptake. In order to get insight in the effective inhibitory concentrations of rLPI, phagocytosis of labelled S. aureus was performed in 10% human serum and with increasing concentrations of rLPI. This figure shows that rLPI has a half-maximum inhibitory concentration (IC50) of 0.3 μg/ml.

### 3.2 IgG-Mediated Phagocytosis

### Materials and Methods

In order to pinpoint whether the observed anti-phagocytic effects of rLPI were mediated by inhibition of complement- or Fcγ-receptor mediated uptake, human sera were replaced for purified IgG during phagocytosis. Human IgG was purified from serum of a healthy volunteer by protein G-affinity chromatography (Amersham Biosciences, Upsalla, Sweden, according to manufacturers instructions) as previously described by Troelstra et al. (Troelstra et al., J. Leukocyte Biol. 61, 173-178 (1997)). IgG was added in various concentrations. Recombinant LPI was tested in a concentration of 8 μg/ml. Phagocytosis was performed for 15 minutes at 37° C. Bacteria were mixed with neutrophils in a ratio of 15:1. Samples were evaluated by flow cytometry and phagocytosis was depicted as the mean bacterial uptake by neutrophils.

### Results

FIG. 7 shows no effect of 8 μg/ml rLPI on the phagocytosis of S. aureus when this was solely mediated by Fcγ-receptors on neutrophils. This was studied by incubating bacteria and neutrophils with purified human immunoglobulins instead of serum.

### 3.3 In a Similar Phagocytosis Experiment rLPI was Compared to the Larger N-Terminally Extended His-Tagged LPI Molecule

### Results

FIG. 8 depicts the inhibitory effect of both rLPI and rHIS-LPI. Both molecules show comparable inhibition at equal concentrations. It was concluded that the presence of a Histidine Tag at the N-terminus of the protein does not interfere with LPI activity.

### Example 4

### 4.1 C3b Deposition on S. aureus

Activation of complement leads to deposition of C3b and C3bi molecules at the bacterium that are recognized by complement receptors on phagocytes. To study whether LPI is an inhibitor of complement activation an assay was developed to measure C3b deposition at the surface of S. aureus.

### Materials and Methods

A stationary growth culture of Cowan EMS was washed three times in PBS. 100 ml of 3×10⁷ bacteria/ml were incubated in 10% human sera at 37° C. Bacteria were washed and surface-bound C3b was detected by incubating bacteria with Fluorescein-conjugated Goat F(ab′)₂ anti-human C3 (Protos immunoresearch, Diessen, The Netherlands) reacting with intact C3b molecules. Then fluorescence per particle was measured using flow cytometry.

### Results

FIG. 9 presents the inhibitory effect of 3 μg/ml rLPI on the deposition of C3b on the surface of S. aureus. C3b deposition was performed by incubation of S. aureus in 10% human serum in the presence of rLPI for varying time intervals. Bacteria were washed and the amount of C3b on the surface was detected by specific labelled antibodies. C3b deposition is depicted as the mean bacterial fluorescence. Heat-inactivated serum, where complement activity is destroyed, was used to control for antibody specificity.

### Example 5

### Complement Activation by Different Pathways

Activation of complement on microorganisms can be initiated via three pathways: the classical, lectin and alternative pathway. In order to recognize all the different micro-organisms we encounter, these pathways act together to activate complement. To study whether the complement-inhibitory role of LPI was due to inhibition of (one of) these three pathways, LPI was tested in 3 well-described assays that specifically measure these pathways

### 5.1 The Classical Pathway (CP)

The CP is activated when C1q binds to antigen-antibody complexes. C1q circulates in serum with two attached serine proteases, C1r and C1s. Upon binding of the C1 complex to an antibody, C1r and C1s are activated. C1s cleaves complement C2 and C4 generating a C4b2a complex that serves as the C3 convertase.

### Materials and Methods

To measure the classical pathway activation route, CH50 measurements were performed similar to Klerx et al. (Klerx et al., J Immunol Methods. 1983, 63:215-20). In short, sheep erythrocytes were incubated with anti-sheep erythrocyte antibodies. Then Ab-covered erythrocytes were incubated in human serum for 1 hour at 37° C. Classical pathway mediated complement activation will lead to formation of membrane attack complexes that lyse the erythrocytes. 10 μg/ml of rLPI were added to test the role of LPI in the classical pathway.

### Results

FIG. 10 shows the lack of effect of LPI on classical pathway complement activation as determined by a hemolysis assay with immunoglobulins coated erythrocytes. LPI does not inhibit the classical pathway.

### 5.2 The Lectin Pathway

The lectin pathway is initiated when MBL or ficolins recognize sugars on microbial surfaces. MBL and ficolin are also complexed with serine proteases called MASPs. MASP-2 is responsible for cleaving O₂ and C4 to generate a C3 convertase.

### Materials and Methods

Lectin pathway-mediated C3 deposition was performed on mannan coated plates as described by Roos et al. (Roos et al., Molecular Immunology Volume 39, Issue 11, January 2003, Pages 655-668). Briefly, C1q-depleted serum was prepared using serum of a healthy donor and a rabbit IgG anti-human C1q coupled to Biogel A5. C1q-depleted serum was incubated on mannan, an efficient activator of MBL-MASPs, for 1 hour at 37° C. C3b deposition was measured by digoxigenin-conjugated anti-human C3 followed by HRP-conjugated sheep anti-dig antibodies.

### Results

FIG. 11 shows the inhibitory effect of rLPI on lectin pathway activation using an ELISA based method on mannan-coated plates and C1q depleted serum with C3b deposition as a readout. LPI strongly inhibits the lectin pathway of complement activation.

### 5.3 The Alternative Pathway

### Materials and Methods

To test the effect of LPI on the alternative pathway, two assays were used. In FIG. 12A, a method was used as described by Schreiber et al. (Schreiber et al, Proc. Natl. Acad. Sci. USA, Vol. 75, No. 8, pp. 3948-3954).

In short, the complement components C3, factor D, factor B and properdin (factor P) were purified to homogeneity. Then 0.5 mg zymosan was incubated with these purified proteins in concentrations similar to that in 25% serum. After 20, 30 or 40 minutes at 37° C., C3b deposition was evaluated by flow cytometry as described in 4.1.

In FIG. 12B an alternative pathway hemolysis assay was used as previously described by Klerx et al. (supra). Briefly, rabbit erythrocytes were suspended in EGTA-VB and incubated with human serum in the presence or absence of recombinant proteins for one hour at 37° C. Erythrocytes were pelleted and 50 μl of supernatant was lysed in 100 μl of water to monitor percentage of haemolysis.

### Results

FIG. 12A shows the lack of inhibition of rLPI on C3b deposition by flow cytometric analysis of zymosan using only purified C3, factor D, factor B and properdin (factor P). FIG. 12B shows that the hemolytic assay is inhibited by LPI, LPI-B and LPI-C(CHIPS is the negative control protein here). With purified components we see no influence of LPI on the alternative pathway.

### Example 6

### 6.1 LPI-Binding

The above-described results have suggested a role for LPI in preventing C3b deposition and subsequent phagocytosis by inhibition of lectin-mediated complement activation. LPI binding experiments on S. aureus were performed to get more information about how LPI might work as an inhibitor of complement activation.

### Materials and Methods

Recombinant LPI was FITC-labelled by incubating 400 μg/ml rLPI in a 0.1 M sodium carbonate buffer (pH9.6). FITC-labelled rLPI was purified from free FITC using a Fast Desalting column (Amersham Biosciences, Upsalla Sweden). 0.5 mg of washed zymosan incubated in 0% or 10% human sera at 0° C. and at 37° C. Also, human sera were depleted from polysaccharide-binding molecules by incubation of 500 ml serum with staphylococcal cell wall homogenate. LPI binding on zymosan in the presence of this lectin-depleted serum was compared with the binding to normal serum at 37° C. After incubation in serum, bacteria were washed and surface-bound rLPI-FITC was measured by flow cytometry.

### Results

FIG. 13 shows a dose dependent association of LPI-FITC to zymosan particles in flow cytometry. The binding is dependent both on the presence of serum and does not proceed at 0° C. FIG. 14 presents the binding kinetics of rLPI on zymosan. Recombinant LPI-FITC binding is dependent on the presence of human serum, because rLPI does not bind bacteria alone. Moreover, rLPI does not bind to bacteria at 0° C. An activation process seems to be necessary. Compared to rLPI binding in human serum, this binding is completely abolished in lectin-depleted serum. This latter result again demonstrates that LPI affects the lectin route of complement activation and not the classical pathway. rLPI-FITC binding is depicted as the mean bacterial fluorescence. From this it is likely that LPI needs a serum component for its interaction and that this component is activation dependent. Because this component is part of the lectin pathway MASP-2 is the most likely candidate.

### Example 7

What Lectin pathway component is inhibited by LPI? 7.1 MBL

The first component in the lectin pathway is MBL. To evaluate the effects of LPI on MBL binding we analyzed the binding of MBL to Zymosan in the presence or absence of rLPI.

### Materials and Methods

0.5 mg of washed zymosan was incubated with 30% of normal human serum or in C3-deficient serum (Sigma) for 30 minutes at 0° C. or 37° C. respectively. MBL was detected by subsequent incubation of zymosan particles with monoclonal anti-MBL antibodies (Hbt, Uden, The Netherlands) followed by Fluorescein conjugated goat-anti-mouse antibodies. Fluorescence per particle was determined by flow cytometric analysis.

### Results

FIG. 15 depicts the binding of MBL to zymosan particles in the presence and absence of LPI. MBL was detected by flow cytometry after staining with a specific anti-MBL antibody. MBL association is not affected by the presence of LPI.

The next step in the lectin pathway involves the MBL associated serine protease-2 (MASP-2). The interaction of LPI with MASP-2 was evaluated in two ways: association and functional inhibition.

### 7.2 MASP-2 Association

### Material and Methods

rLPI was labelled with ¹²⁵I using Iodogen (1,3,4,6-tetrachloro-3a,6a-diphenylglycoluril) (Sigma) as the oxidising agent (Fraker and Speck, 1978). Free 125-iodide was removed by desalting on a PD-10 Sephadex G-25 gel filtration column (Pharmacia) which was presaturated with 0.1% (v/v) Emulphogene BC720 [Sigma] in HBS. Radiolabelled protein fractions were pooled and stored at 4° C. ¹²⁵I-LPI was incubated with purified complement proteins, including recombinant MASP-1 and MASP-2 (consisting of the two complement control regions and the serine protease domains as described by Ambrus et al., 2003. G. J. Immunol. 170 (2003), pp. 1374-1382) for 15 minutes at 37° C. Protein mixtures were run on a Superose-6 column (Amersham) and specific activity of ¹²⁵I-LPI was determined by measuring collected fractions on a Mini-Assay type 6-20 manual g counter (Mini Instruments, Burnham-on-Crouch, Essex, UK).

### Results

FIG. 16 shows the association between of rLPI and rMASP-2 as analyzed by size-exclusion chromatography using iodinated rLPI. LPI and MASP-2 do interact to form a larger sized molecular complex.

### 7.3 MASP-2 Activity

### Material and Methods

0.2 mg rMASP-2 was incubated with 0.1 mg Prothrombin (Calbiochem) and 0.1 mM VPR-AMC (Bachem, Bubendorf, Switzerland) in the presence or absence of various concentrations of rLPI. rMASP-2 will cleave Prothrombin into thrombin. Cleavage of the thrombin specific substrate VPR-AMC was measured every 30 seconds for 1 hour using a microtitre plate reader (Fluoroskan, Thermo Life Sciences, Basingstoke, UK) exciting samples at 355 nm and reading emission at 460 nm.

### Results

FIG. 17 shows the inhibition of rMASP-2 activity by rLPI, in a specific proteolytic cleavage assay, using a fluorescent substrate. LPI does inhibit MASP-2 activity.

### Example 8

### 8.1 The Molecular Mechanism of LPI

MASP-2 has two specific proteolytic activities: the cleavage of C2 and the cleavage of C4. To determine what the exact mechanism of action of LPI is both actions of MASP-2 were tested and the effects of LPI on these separate steps evaluated.

### Materials and Methods

### C4 Deposition

LP-mediated C4 deposition was measured exactly as described in section 5.2 for measuring LP-mediated C3 deposition with the exception that a monoclonal antibody against C4 was used in order to detect C4b deposition. Alternatively, MBL-MASP complexes were captured on mannan coated plates by incubating 100 μl of 50% human serum in 1 M NaCl buffer (to prevent C1q binding to IgG). After washing MBL-MASP complex in low salt buffers, purified human C4 was added and MBL-MASP mediated cleavage of C4 was allowed for 1 hour at 37° C. Deposited C4b molecules were detected as described above.

### C2 Cleavage Assay

Activation of C2 as a result of incubating human serum with zymosan particles was assayed as following. 0.25 mg of zymosan was incubated with 40% human serum for 0, 20 or 40 minutes. Cleaved and uncleaved C2 molecules were detected by subjecting 10% of the incubated serum to gel electrophoresis. Western blotting using goat anti-human C2 (Quidel, San Diego, Calif.) followed by HRP conjugated Donkey anti-goat antibodies and ECL (Amersham).

### Results

FIG. 18 shows LP-mediated C4b deposition on mannan-coated plates in the presence of increasing amounts of rLPI and different amounts of serum. No inhibition is observed. In FIG. 19 C4 activation by captured MBL-MASP complexes cleaving purified human C4 is depicted. The effect of the addition of LPI is compared to that of an anti-MBL antibody (total inhibition). LPI shows no inhibition of MASP-2 dependent C4 deposition. The C2 cleavage however is clearly affected by LPI.

In FIG. 20 the C2 cleavage into C2a and C2b was analysed by Western blotting after complement activation had preceded in serum at various time periods with or without LPI. FIG. 20a shows the blot after staining with anti-C2. FIG. 20b shows the densitometric analysis of this same blot for the specific C2b band. LPI clearly inhibits C2 cleavage.

Taken together LPI is designed to interfere with MASP-2 mediated cleavage of C2, thereby blocking the entire lectin pathway (MBL and ficolins are all dependent on MASP-2). Furthermore, this action makes LPI also completely specific for the lectin pathway, without interfering with other complement pathways.

### Example 9

### The LPI Activity of LPI-Like Molecules LPI-B and LPI-C

### 9.1. In a Separate Phagocytosis Experiment LPI-B and LPI-C were Tested for LPI-Activity

### Materials and Methods

The phagocytosis assay was performed as described in 3.1.

### Results

FIG. 21 depicts the anti-phagocytic effect of both rLPI-B and LPI-C in the presence of different concentrations of serum. From this we conclude that LPI-B and LPI-C have at least LPI activity.

1.-48. (canceled)

49. A method of treating or preventing an acute or chronic inflammatory reaction in a subject, the method comprising administering a peptide or polypeptide to said subject for the treatment of said acute and chronic inflammatory reaction in said subject,
wherein said peptide or polypeptide is encoded by a nucleotide sequence corresponding to a sequence selected from:
a) a nucleotide sequence comprising a fragment of the sequence set forth in SEQ ID NO:1;
b) a nucleotide sequence having at least about 90% nucleotide sequence identity to the nucleotide sequence set forth in SEQ ID NO:1;
c) a nucleotide sequence encoding a peptide or polypeptide comprising the amino acid set forth in SEQ ID NO:3;
d) a nucleotide sequence that hybridizes under stringent hybridization to any one of the nucleotide sequences (a), (b), or (c); and
e) a nucleotide sequence complementary to any one of the nucleotide sequences (a), (b), (c), or (d);
and wherein the peptide or polypeptide has lectin pathway inhibitory (LPI) activity and reduces mannose binding lectin associated serine protease-2 (MASP2) dependent cleavage of complement protein C2 into C2a and C2b.

50. The method of claim 49, wherein the nucleotide sequence comprising a fragment of the sequence set forth in SEQ ID NO:1 corresponds to nucleotides 1-490 of SEQ ID NO:1.

51. The method of claim 49, wherein the nucleotide sequence comprising a fragment of the sequence set forth in SEQ ID NO:1 corresponds to nucleotides 41-490 of SEQ ID NO:1.

52. The method of claim 49, wherein the nucleotide sequence comprising a fragment of the sequence set forth in SEQ ID NO:1 corresponds to nucleotides 125-490 of SEQ ID NO:1.

53. The method of claim 49, wherein the nucleotide sequence has at least about 90% nucleotide sequence identity to the nucleotide sequence set forth in SEQ ID NO:1.

54. The method of claim 49, wherein the stringent hybridization conditions comprise overnight hybridization at 42° C. in 5×SSC and washing at 65° C. in 0.1×SSC.

55. The method of claim 49, wherein the peptide or polypeptide is administered in a therapeutic composition.


SEQUENCE LISTING (20 sequences)

SEQ ID NO: 1 - DNA, 510 bp, Staphylococcus aureus
  misc_feature: Figure 2a, LPI gene of NCTC 8325
atctatatagttaatgaataattaatgtacttttttttagttagtcattaaaataaattagtactaattactaaggagaataaaaaatgaaaattagaaaatctatacttgcgggaactttagcaatcgttttagcatcaccactagtaactaatctagataaaaatgaggcacaagctagcacaagcttgccaacatcgaatgaatatcaaaacgaaaagttagctaatgaattaaaatcgttattagatgaactaaatgttaatgaattagctactggaagtttaaacacttattataagcgaactataaaaatttcaggtcaaaaagcaatgtatgctcttaagtcaaaagactttaagaaaatgtcagaagcaaaatatcaacttcaaaagatttataacgaaattgacgaagcactaaaaagtaaatattaaaaaaaccacccgtaaaagggtggttttaattttctagataatataaaagtgttcataaataaaacagtatagg

SEQ ID NO: 2 - DNA, 510 bp, Staphylococcus aureus
  misc_feature: Figure 2a, LPI gene of N315
atctatatagttaatgaataattaatgtacttttttttagttagtcattaaaataaattagtactaattactaaggagaataaaaaatgaaaattagaaaatctatacttgcgggaactttagcaatcgttttagcatcaccactagtaactaatctagataaaaatgaggcacaagctagcacaagcttgccaacatcgaatgaatatcaaaacgaaaagttagctaatgaattaaaatcgttattagatgaactaaatgttaatgaattagctactggaagtttaaacacttattataagcgaactataaaaatttcaggtctaaaagcaatgtatgctcttaagtcaaaagactttaagaaaatgtcagaagcaaaatatcaacttcaaaagatttataacgaaattgacgaagcactaaaaagtaaatattaaaaaaaccacccgtaaaagggtggttttaattttctagataatataaaagtgttcataaataaaacagtatagg

SEQ ID NO: 3 - protein, 116 aa, Staphylococcus aureus
  misc_feature: Figure 3, LPI protein of NCTC 8325
MKIRKSILAGTLAIVLASPLVTNLDKNEAQASTSLPTSNEYQNEKLANELKSLLDELNVNELATGSLNTYYKRTIKISGQKAMYALKSKDFKKMSEAKYQLQKIYNEIDEALKSKY

SEQ ID NO: 4 - protein, 116 aa, Staphylococcus aureus
  misc_feature: Figure 3, LPI protein of N315
MKIRKSILAGTLAIVLASPLVTNLDKNEAQASTSLPTSNEYQNEKLANELKSLLDELNVNELATGSLNTYYKRTIKISGLKAMYALKSKDFKKMSEAKYQLQKIYNEIDEALKSKY

SEQ ID NO: 5 - protein, 116 aa, Staphylococcus aureus
  misc_feature: Figure 3, LPI-B
MKFKKYILTGTLALLLSSTGIATIEGNKADASSLDKYLTESQFHDKRIAEELRTLLNKSNVYALAAGSLNPYYKRTIMMNEYRAKAALKKNDFVSMADAKVALEKIYKEIDEIINR

SEQ ID NO: 6 - protein, 116 aa, Staphylococcus aureus
  misc_feature: Figure 3, LPI-C
MKFKKYIVAGTLAVLLSTTAVSTLDGNKADASSKKDYIIQSEFHDKRIAEELKSLLDQSYVNDLAAGSLNPYYKRMIMMNQYRAKAALKSNNFAKMAEAKVGLENIYKEIDEIINR

SEQ ID NO: 7 - protein, 114 aa, Staphylococcus aureus
  misc_feature: Figure 3, LPI-D protein of NCTC 8325
MTTQMKIKTYLVAGIKAALLDTTGIKLASKSETTSHTYQHQALVDQLHELIANTDLNKLSYLNLDAFQKRDILAAHYIAKSAIRTKNLDQMTKAKQRLESIYNSISNPLHSQNN

SEQ ID NO: 8 - protein, 114 aa, Staphylococcus aureus
  misc_feature: Figure 3, LPI-D protein of N315
MTTQMKIKTYLVAGIKAALLDTTGIKLASKSETTSHTYQHQALVDQLHELIANTDLNKLSYLNLDAFQKRDILAAHYIAKSAIRTKNLDQMTKAKHRLESIYDSISNPLHSQNN

SEQ ID NO: 9 - DNA, 364 bp, Staphylococcus aureus
  misc_feature: Figure 2b, LPI-B gene
ggagagtttacaatgaaatttaaaaaatatatattaacaggaacattagcattacttttatcatcaactgggatagcaactatagaagggaataaagcagatgcaagtagtctggacaaatatttaactgaaagtcagtttcatgataaacgcatagcagaagaattaagaactttacttaacaaatcgaatgtatatgcattagctgcaggaagcttaaatccatattataaacgtacgattatgatgaatgaatatagagctaaagcggcacttaagaaaaatgatttcgtatcaatggctgatgctaaagttgcattagaaaaaatatacaaagaaattgatgaaattataaatagataat

SEQ ID NO: 10 - DNA, 364 bp, Staphylococcus aureus
  misc_feature: Figure 2b, LPI-C gene
ggagaatttacaatgaaatttaaaaaatatatagtagcaggaacattagcagtactattatcaacaacagcagtatcaacgttagatgggaataaagcagatgcaagtagtaagaaagactatataattcaaagtgagtttcatgataaacgaattgctgaagaattgaaatcattacttgatcaatcttatgtaaatgatttagctgcaggaagcttaaacccatactacaaacgtatgattatgatgaaccaatatagagcaaaagcagcactaaaaagtaataatttcgcaaaaatggctgaagctaaagttggattagaaaacatttacaaagaaattgatgaaattataaatagataat

SEQ ID NO: 11 - DNA, 365 bp, Staphylococcus aureus
  misc_feature: Figure 2b, LPI-D gene of NCTC 8325
ggagtaacaaagcatgacaacacaaatgaaaatcaaaacatatttagttgctggtattaaagcggcgctccttgatacgactggtattaaattagcaagcaaatctgaaactacatcacatacgtatcaacatcaagcgcttgtagatcaattacatgaattaatagcaaacactgacttaaataaattatcgtacctaaatttagatgcgtttcaaaaacgcgatattttagctgcgcactatattgcaaaatccgctatacgcactaaaaatttggatcaaatgactaaagcgaaacaaagattagaaagtatttacaattcaatttctaaccctttgcattcacaaaacaattaataattca

SEQ ID NO: 12 - DNA, 365 bp, Staphylococcus aureus
  misc_feature: Figure 2b, LPI-D gene of N315
ggagtaacaaagcatgacaacacaaatgaaaatcaaaacatatttagttgctggtattaaagcggcgctccttgatacgactggtattaaattagcaagcaaatctgaaactacatcacatacgtatcaacatcaagcgcttgtagatcaattacatgaattaatagcaaacactgacttaaataaattatcgtacctaaatttagatgcgtttcaaaaacgcgatattttagctgcgcactatattgcaaaatccgctatacgcactaaaaatttggatcaaatgactaaagcgaaacatagattagaaagtatttacgattcaatttctaaccctttgcattcacaaaacaattaataattca

SEQ ID NO: 13 - DNA, 15 bp, Artificial Sequence
  pRSET-B vector enterokinase recognition site
gacgatgacgataag

SEQ ID NO: 14 - DNA, 15 bp, Artificial Sequence
  pRSET-B vector enterokinase recognition site mutated
gacgatgacgacaag

SEQ ID NO: 15 - DNA, 22 bp, Artificial Sequence
  primer LPI-5′
gagcacaagcttgccaacatcg

SEQ ID NO: 16 - DNA, 30 bp, Artificial Sequence
  primer LPI-3′
ccggaattcttaatatttactttttagtgc

SEQ ID NO: 17 - DNA, 21 bp, Artificial Sequence
  primer LPI-B-5′
gagtagtctggacaaatattt

SEQ ID NO: 18 - DNA, 29 bp, Artificial Sequence
  primer LPI-B-3′
ccggaattcttatctatttataatttcat

SEQ ID NO: 19 - DNA, 21 bp, Artificial Sequence
  primer LPI-C-5′
gagtagtaagaaagactatat

SEQ ID NO: 20 - DNA, 27 bp, Artificial Sequence
  primer LPI-C-3′
ggaattccttatctatttataatttca